SEQUENCE LISTING (47 sequences)

SEQ ID NO: 1 - DNA, 26 bp, Artificial Sequence
  Primer S1
ggatccgtgcttgggcgagagtagta

SEQ ID NO: 2 - DNA, 26 bp, Artificial Sequence
  Primer AS1
ggatccttagtgctggtatgatcgca

SEQ ID NO: 3 - DNA, 20 bp, Artificial Sequence
  Primer 5S2F
gtgcttgggcgagagtagta

SEQ ID NO: 4 - DNA, 19 bp, Artificial Sequence
  Primer 5S2R
ttagtgctggtatgatcgc

SEQ ID NO: 5 - DNA, 655 bp, Asparagus filicinus
  5S rDNA Spacer clone a
gtgcttgggcgagagtagtactaggatgggtgacctcctgggaagtcctcgtgttgcacccctcctttttgcccggcgcgcaaattgcgactgagagcacgtttaattttattttattattttccgccaatcggcggctcctttttttaccccctcatttcctcccgccgtcagcgcggacgtctgggaatgagaagatgagaagcacgtcgggcttctcgttgaacaaggtttcggggagggggctgaccgtcgggcaacttagccccctccggatcggaagttgcgggtcggggaggtcacgcggaagcccgtccggcggaattggagtgtttccccgcgctcggggcaacgtcagggggtccgggccagcgtttcccttgtgagcaataaaaattgcctttatccctggcaggtacaaaaacgcagatatccctcaaaccgtaagggatttggcgaaacggaggaagcgaacgaacccttcgggttcgttcggttggctccgtctcgccgagataagcgattttcatatataattcgtccaattccgacttacggcttgaaagttacgccctattctccgatctatgttcagaacctcgcctaaggggggaaggagacggctagtggatgggtgcgatcataccagcactaa

SEQ ID NO: 6 - DNA, 658 bp, Asparagus filicinus
  5S rDNA Spacer clone b
gtgcttgggcgagagtagtactaggatgggtgacctcctgggaagtcctcgtgttgcacccctcctttttgctcggcgcgcaaattacgactgagagcccgtttaattttatttcattattttccgccaatcggcggctcctttttttaccccctcatttcttcccgccgtcagggcggacgcctgggaacgagaagatgagaagcacgtcgggcttctcgttgaacaaggtttcgcggagggggccgaccgtcgggcaactacgtccccccccccggatcggaagttgcgggtcggggaggtcacgcggaagcccgtccggcggaattggagtgtttccccgcgctcggggcaacggctgggggtccgggccagcgtttccctcgtaggcaataaaaattgcctttatccctagcagagacaaaaacgcggatatccctcaaaccgtaagggatttggcgaaacggaggaagcgaacgaacccttcgggttagttcggttggctccgtctcgtcgagataagcgattttcgtatataattcgtccaattccgacttacggtttgaaagttacgccctattcttcgatctatgttcggagcctcgcctaaggggggaaggagacggcgagtggatgggtgcgatcataccagcactaa

SEQ ID NO: 7 - DNA, 464 bp, Stemona collinsae (Specimen G57)
  5S rDNA Spacer clone a
gtgcttgggcgagagtagtactaggatgggtgacctcctgggaagtcctcgtgttgcacccctttaacttttgcagcgcccgcccgtcacgtctcgcagaaaactcggggtcgatttgtgggaacggaaatccctcgattctgacttgttagggtgtgaatttggagaattcgacaccgccgacggcctgcggctgtcggccgcgccgttgctctcagaatcagctttccgtggacaggtaattcgtccgaaacggacacccgtagctcccgcaccgtcgattccagccgagtcgcgcaattcggctccaaaatacggcagcacacgccgatcccacccttgttcctcgtaattttagcctcgggagcccctcgcgcgtcgtgacagccttggtgggtgcaggtgcgcgaggttaaaacggaaatgcgtcgcacgtgtgtcgggtgcgatcataccagcactaa

SEQ ID NO: 8 - DNA, 465 bp, Stemona collinsae (Specimen G57)
  5S rDNA Spacer clone b
gtgcttgggcgagagtagtactaggatgggtgacctcctgggaagtcctcgtgttgcacccctttaacttttgcagcgcccgcgcgtgacgttttgcagaaaactagggtgtcgattcgcgggaaaggaaatccctcgattctgacttgttagggtttgaatttggagaattcgacaccgccgacggcctgcggctgtcggccgcgccgtagctctcagaatcagctttccgtggacaggtaattcgtccgaaacggacacccgtagctcccgcaccgtcgattccaaccgagtcgcgcaattcggctccgaaatacggcagcactctcccatcccacccttgttcctcgtaattttatcctcgggagcccctcgcgcgtcgtgaaagccttggtgggggcgggtgcgcgaggttaaaacggaaatgcgtcgcacgtgtgtcgggtgcgatcataccagcactaa

SEQ ID NO: 9 - DNA, 465 bp, Stemona collinsae (Specimen G57)
  5S rDNA Spacer clone c
gtgcttgggcgagagtagtactaggatgggtgacctcctgggaagtcctcgtgttgcacccctttaacttttgcagcgcccgcgcgtgacgttttgcagaaaactagggtgtcgatttgcgggaagggaaatccctcgattctgacgtgttagggtgggaatttggagaattcgacaccgccgacggcctgcggcggtcggccgcgtcgtggctctcagaatcagctttccgtggacaggtaattcgtccgaaacggacacccgtagctcccgcaccgttgattccagccgagtcgcgcaattcgtctccgaaacacggcagcactcggcgatcccaccctttttcctcgtaattttagccccgagcgcccctcgcgcgtcgtggaagcgttcgtgggcgcgggtgcgcgaggttgaaacggaaatgcgacgcacctgtgtcgggtgcgatcataccagcactaa

SEQ ID NO: 10 - DNA, 477 bp, Stemona japonica (Specimen Hu & But23971)
  5S rDNA Spacer clone a
gtgcttgggcgagagtagtactaggatgggtgacctcctgggaagtcctcgtgttgcaccccttttcctttttgagcacgggcatattaagttttgcagaaaaatggagagcggctgccgaaaacacgaatccgtcgtcgttccggacgggttagagatgggatccgaggtaatcctcgcctccggccgcctgcggtcgaagtccaagtcgtaggtctctggatgggccttccgtcgggaggtgattcgtccgaaacgaacagccatagctcccgcggcgccgattttagtggcgctagttttattattagtatactatttttatttttatttttgtttttatttttttttgggggggggggatttatggcctctcccagccactcgagcgtcgttcatggaatcgggattgcgaatggacagggttaagagggaaacgcggtagaaaaatgtcgggtgcgatcataccagcactaa

SEQ ID NO: 11 - DNA, 465 bp, Stemona japonica (Specimen Hu & But23971)
  5S rDNA Spacer clone b
gtgcttgggcgagagtagtactaggatgggtgacctcctgggaagtcctcgtgttgcaccccttttcctttttgatcaagggcatattaagttttgcagaaaaatggagagcggttgtcgaaaacacgaatccgtcgtcgttccggacaggttagagatgggatttgaggtaatcctcgcctccggccgcctgcggtcgaagtccacgttgtaggtctctggatgggctttccgtcgggaggtgattcgtccgaagcggacagccgtagctcccgcggcgccgattttagtggcgccagttttattattagtatactatttttatttttatttttgttttttttggggggatttatggcctctcccagccactcgagcgtcgttcatggaatcaggattgagaagggacagggttaagagggaaacgcggtagaaaaatgtcgggtgcgatcataccagcactaa

SEQ ID NO: 12 - DNA, 478 bp, Stemona japonica (Specimen Hu & But24032)
  5S rDNA Spacer clone a
gtgcttgggcgagagtagtactaggatgggtgacctcctgggaagtcctcgtgttgcaccccttttcctttttgagcacgggcatattaagttttgcagaaaaatggagagcggttgccgaaaacacgaatccgtcgtcgttccggacgggttagagatgggatctgaggtaatcctcgcctccggccgcctgcggtcgaagtccaagtcgtaggtctctggatgggctttccgtcgggaggtgattcgtccgaaacgaacagccatagctcccgcggcgccgattttagtggcgctagttttattattagtatactatttttatttttatttttgtttttatttttttttggggggggggggatttatggcctctcccagccactcgagcgtcgttcatggaatcgggattgcgaatggacagggttaagagggaaacgcggtagaaaaatgtcgggtgcgatcataccagcactaa

SEQ ID NO: 13 - DNA, 477 bp, Stemona japonica (specimen ICM20042543)
  5S rDNA Spacer clone b
gtgcttgggcgagagtagtactaggatgggtgacctcctgggaagtcctcgtgttgcaccccttttcctttttgagcacgggcatattaagttttgcagaaaaatggagagcggttgccgaaaacacgaatccgtcgtcgttccggacgggttagagatgggatctgaggtaatcctcgcctccggccgcctgcggtcgaagtccaagtcgtaggtctctggatgggctttccgtcgggaggtgattcgtccgaaacgaacagccatagctcccgcggcgccgattttagtggcgctagttttattattagtatactatttttatttttatttttgtttttatttttttttgggggggggggatttatggcctctcccagccactcgagcgtcgttcatggaatcgggattgcgaatggacagggttaagagggaaacgcggtagaaaaatgtcgggtgcgatcataccagcactaa

SEQ ID NO: 14 - DNA, 464 bp, Stemona japonica (specimen ICM20042543)
  5S rDNA Spacer clone c
gtgcttgggcgagagtagtactaggatgggtgacctcctgggaagtcctcgtgttgcaccccttttcctttttgagcacgggcatattaagttttgcagaaaaatggagcgcggttgccgaaaacacgaatccgtcgtcgttccggacgggttagagatgagatctgaggtaagcctcgcctccggccgcctgcggtcgaagtccacgtcgtaggtctctggatgggctttccgtcgggaggtgattcgtccgaaaaggacagccgtagctcccgcggcgccgattttagtggcgccagttttattattagtatactatttttatttttatttttatttttttggggggatttatggcctctcccagccactcgagcgtcgttcatggaatcgggattgcgaagggacagtgttaagagggaaatgcggtagaaaaatgtcgtgtgcgatcataccagcactaa

SEQ ID NO: 15 - DNA, 466 bp, Stemona kerrii (Specimen 15)
  5S rDNA Spacer clone a
gtgcttgggcgagagtagtactaggatgggtgaccttctgggaagtcctcgtgttgcacccgttttgatttttgggccacgcggatgctcagctttgaagaaaaccgcagcgagagtgccgaaagcacggatccatcgattcggacgcgttagagatggaatttggcgaaattctcgtcgccgaccgccttcggccgtcggccacgtcgtaggtctctggatcggctttccgtcgacagctaattcgtccgaaacggacacccgtagctcccgcagcgtcgattttcgtggagtggcgcaatccggcacccgattccaactataagccgtgcggtttctttccttttttgtggattttgtcccccgaggccacccgaccgtcggtcttggaatcgggatcgcgggggaaccgggttaagatggcaaagcggaccaaacatgtcgtgtgcgatcataccagcactaa

SEQ ID NO: 16 - DNA, 466 bp, Stemona kerrii (Specimen 15)
  5S rDNA Spacer clone b
gtgcttgggcgagagtagtactaggatgggtgacctcctgtgaagtccacgtattgcacccgttttgatttttgggccacgcggatgctcagctttgaggaaaacggcagcgagagtgccgaaagcacggatccgtcgattcggacgtgttagagatggaatttggcgaaattctcgtcgctgaccgccttcggccttgggccacgtcgtaggtctctggatcggctttccgtcgacagctaattcgtccgaaacggacacccgtggctcccgcagcgtcgattttcgtggagtggcgcaatccggcgcccgattccaactatttgccgtgcattttctttccttttttgtggattttgtcccccgaggccacccgaccgtcggtcttggaatcgggatcgcgggggaaccgggttaagatggcaaagcggaccaaacgtgtcgtgtgcgatcataccagcactaa

SEQ ID NO: 17 - DNA, 466 bp, Stemona kerrii (Specimen 15)
  5S rDNA Spacer clone c
gtgcttgggcgagagtagtactaggatgggtgacctcctgggaagtccttgtgttgcacccgttttgatttttgggccatgcggatgctcagctttgaagaaaatcgcagcgagagtgccgaaagcacggatccatcgattcggacgcgttagagatggaatttggcgaaattctcatcgccaaccgccttcggccgtcggccacgtcgtaggtctctggatcggctttccgtcgacagctaattcgtccgaaacggacacccgtagctcccgcagcgtcgattttcgtggagtggcgcaatccggcacccgattccaactatacgccgtgcattttcttcccttttttgtggattttgtcccccgaggccacccgaccgtcggtcttggaatcgggatcgcgggggaaccgggttaagatggcaaagcggacaaaacatgtcgtgtgcgatcataccagcactaa

SEQ ID NO: 18 - DNA, 466 bp, Stemona kerrii (specimen 32)
  5S rDNA Spacer clone a
gtgcttgggcgagagtagtactaggatgggtgacctcctgggaagtcctcgtgttgcacccgttttgctttttgggccacgcggatgttcagctttgtagaaaaccgcagcgagagtgccgaaagcacggatccatcgattcagacgtgttagagatggaatttggcgaaattctcgtcgccgaccgccttcggccgtcggccacgtcgtaggtctctggatcggctttccgtcaacgggtaattcgtccgaaacggacacccgtagctcccgcagcgtcgattttcgtggagtggcgcaatccggcgcccgattccaactatacgccgtgcattttctttccttttttgtggattttgtcccacgaagccacccgaccgtcggtctgggaatcgggatcgcgagcgaaccgggttaagatggcaaagcggaccacacatgtcgtgtgcgatcataccagcactaa

SEQ ID NO: 19 - DNA, 466 bp, Stemona kerrii (specimen 32)
  5S rDNA Spacer clone b
gtgcttgggcgagagtagtactaggatgggtgacctcctgggaagtcttcgtgttgcacccgttttgctttttgggccacgcggatgctcagctttgaggaaaaccgcagcgagagtgccgaaagcacggatccatcgattcggacgtgttagagatggaatttggcgaaattctcgtcgctgaccgccttcggccgtcggccacgttgtaggtctctggatcggctttccgtcgacaggtaattcgtccgaaacggacacccgtagctcccgcagcggcgatttccgtggagttgcgcaatacggcgcccgattccaactgtacgccgtgcattttctttccttctttacggattttgtcccccgaagccccccgaccgtcggtcttggaatcgggatcgcgggggaaccgggttaagatggcaaagcggaccaaacatgtcgtgtgcgatcataccagcactaa

SEQ ID NO: 20 - DNA, 466 bp, Stemona kerrii (specimen 32)
  5S rDNA Spacer clone c
gtgcttgggcgagagtagtactagaatgggtgacctcctgggaagtcctcgtgttgcacctgttttgctttttgggccacacggatgctcagctttgaagaaaaccgcagcgagagtgccgaaagcacggatccatcgattcggacgtgttagagatggaatttggcgaaattctcgtcgccgaccgccttcgaccgtcggccacgtcgtaggtctctggatcggctttccgtcgacaggtaattcgtccgaaacggacacccgtagctcccgcagcggcgatttccgtggagttgcgcaatacggcgcccgattccaactgtacgccgtgcattttctttccttctttacggattttgtcccccgaagccccccgaccgtcggtcttggaatcgggatcgcgggggaaccgggttaagatggcaaagcggaccaaacatgtcgtgtgcgatcataccagcactaa

SEQ ID NO: 21 - DNA, 466 bp, Stemona kerrii (specimen 32)
  5S rDNA Spacer clone d
gtgcttgggcgagagtagtactaggattggtgacctcctgggaagtcctcgtgttgcacccgttttgctttttgggccacgcggatgctcagctttgaagaaaaccgcagcgagagtgccgaaagcacggatccatcgattcggacgtgttagagatggaatttggcgaaattctcgtcgccgaccgccttcgaccgtcggccacgtcgtaggtctctggatcggctttccgtcgacaggtaattcgtccgaaacggacacccgtagctcccgcagcggcgatttccgtggagttgcgcaatacggcgcccgattccaactgtacgccgtgcattttctttccttctttacggattttgtcccccgaagccccccgaccgtcggtcttggaatcgggatcgcgggggaaccgggttaagatggcaaagcggaccaaacatgtcgtgtgcgatcataccagcactaa

SEQ ID NO: 22 - DNA, 478 bp, Stemona parviflora (specimen Hu & But 24034)
  5S rDNA Spacer clone a
gtgcttgggcgagagtagtactaggatgggtgacctcctgggaagtcctcgtgttgcaccccttttgtttttggagcactcgaattccacgtttcgaggaacacagtcgcgaggcatccgggaacacaaatccgtcgatcgggacgtgttagggatggattttcatgaaattctggcctccgacggcctgcggctatcggccacgtcgtagatctcccgttcagctttccgttgacaggtaattcgcacgaaacggacacccgtagcccccgcggagtcgattttcgcggggtcgcgcatctcggcgtctcattgcagcgacgcccctcgctttcgttgttgttgttgtttttttttttttgtgaattaacccctcccgggccagtcgatcgtcgttcaaggcatcgggagggcggaagaacattgttgaaagtgaaaagcagccaagtaatgtcggatgcgatcataccagcactaa

SEQ ID NO: 23 - DNA, 483 bp, Stemona parviflora (specimen Ma9066)
  5S rDNA Spacer clone a
gtgctgggcgagagtagtactaggatgggtgacctcctgggaagtcctcgtgttgcaccccttttgtttttggagcacgcgaattccacgtttccaggaacacagtcgcgaggcatccgggagcacaaatccgtcgatccggacgtgttagggatggattttctcgaatttctggcctccgacggcctgcggctgtcggccaagtcgtagatctcccgttccgctttccgttgacaggtaattcgcacgaaacggacacccgtagctcccgctgcgtcgattttcgcagggtcgcgcgtctcggcgtctcattgcagcgacgcccctcgctttcgttgttgtcggatttttcttttttttttttttgtgaattgacccctcccaggcctgtcgatcgtcgctcaaggcatcgggagggcggaagaacattgttaaaaaggaaaagcagccaagtaaggtcggatgcgatcataccagcactaa

SEQ ID NO: 24 - DNA, 480 bp, Stemona parviflora (specimen Ma9066)
  5S rDNA Spacer clone b
gtgcttggtcgagagtagtactagtatgggtgacctcctgggaagtcctcgtgttgcaccccttttgtttttggagcactcgaattccacgtttcgaggaacacagtcgcgaggcatccgggaacacaaatccgtcgatccggacgtgttagggatggattttcatgaaattctggcctccggcggcctgcggctgccggccacgtcgtagatctcccgttcagctttccgttgacaggtaatacgcacgaaacggacacccgtagctcccgcggcgtcgattttcgcggggtcgcccatctcggcgtctcattgcagcgacgcccctcgctttcgttgttgttgttttttttttttttttttgtgaattaacccctccctggccagtcgatcgtcgttcaaggcatcgggagggcggaagaacattgttaaaagtgaaaagcagccaagtaatgtcggatgcgatcataccagcactaa

SEQ ID NO: 25 - DNA, 483 bp, Stemona parviflora (specimen Ma9066)
  5S rDNA Spacer clone c
gtgcttgggcgagagtagtactaggatgggtgacctcctgggaagtcctcgtgttgcaccccttttgtttttggagcacgcgaattccacgtttccaggaacacagtcgcgaggcatccgggagcacaaatccgtcgatccggacgtgttagggatggattttctcgaatttctggcctccgacggcctgcggctgtcggccacgtcgtagatctcccgttccgctttccgttgacatgtaattcgcacgaaacggacacccgtagctcccgctgcgtcgattttcgcagggtcgcgcgtctcggcgtctcattgcagcgacgcacctcgctttcgttgttgtcggatttttctttttttttttttgtgaattgacccctcccaggccagtcgatcgtcgctcaaggcatcgggagggcggaagaacattgttaaaaaggaaaagcagccaagtaaggtcggatgcgatcataccagcactaa

SEQ ID NO: 26 - DNA, 498 bp, Stemona sessilifolia (Hu & But23972)
  5S rDNA Spacer clone a
gtgcttgggcgagagtagtactaggatgggtgacctcctgggaagtcctcgtgttgcaccccttctccttttttcgcacaggaatattaagttttgcagaaaaatacagagcggttggcgaaaacaagaatccgtcgtcgcggacgcgttagagatggaatttgagaaaaattctcgtctccggccgcctgcggtcgaagtccacgtcgtaggtcactggatgggctttccgtcgggaggtaattcgtccgaaacggacacccgtagcccccgcgacgccgattttcgaggcgtcgcgcaatcgtcgccaaattccgtcagcgtgccgtgcctctcctattattactagactatatatccatttttttaaaaaaaacaaaaaaaattatggcctctcccagccagtcgggcgtcgttgatggaatcgggagtgcgatacgtcagggttaagatggaaacgcggtagaaagatgtcgggtgcgatcataccagcactaa

SEQ ID NO: 27 - DNA, 498 bp, Stemona sessilifolia (Hu & But23972); identical to SEQ ID NO: 26
  5S rDNA Spacer clone b

SEQ ID NO: 28 - DNA, 498 bp, Stemona sessilifolia (Hu & But23972)
  5S rDNA Spacer clone c
gtgcttgggcgagagtagtactaggatgggtgacctcctgggaagtcctcgtgttgcaccccttctccttttttcgcacaggaatattaagttttgcagaaaaatacagagcggttggcgaaaacaagaatccgtcgtcgcggacgcgttagagatggaatttgagaaaaattctcgtctccggccgcctgcggtcgaagtccacgtcgtaggtcactggatgggctttccgtcgggaggtaattcgtccgaaacggacacccgtagcccccgcgacgccgattttcgaggcgtcgcgcaatcgtcgccaaattccgtcagcgtgccgtgcctctcctattattactagactatatatccatttttttaaaaaaaacaaaaaaaattatggcctctcccagccagtcgggcgtcgttgatggaatcgggagtgcgataggtcagggttaagatggaaacgcggtagaaagatgtcgggtgcgatcataccagcactaa

SEQ ID NO: 29 - DNA, 498 bp, Stemona sessilifolia (specimen Hu & Yung 606)
  5S rDNA Spacer clone a
gtgcttgggcgagagtagtactaggatgggtgacctcctgggaagtcctcgtgttgcaccccttctccttttttcgcacaggaatattaagttttgcagaaaaatacagagcggttggcgaaaacaagaatccgtcgttgcggacgcgttagagatggaatttgagaaaaattctcgtctccggccgcctgcggtcgaagtccacgtcgtaggtctctggatgggctttccgtcgggaggtaattcgtccgaaacggacacccgtagcccccgcgacgccgattttcgaggcgtcgcgcaatcgtcgccaaattccgtcagcgtgccgtgcctctcctattattactagactatttatccatttttttaaaaaaaacaaaaaaaattatggcctctcccagccagtcgggcgtcgttgatggaatcgggagtgcgatacgtcagggttaagatggaaacgcggtagaaagatgtcgggtgcgatcataccagcactaa

SEQ ID NO: 30 - DNA, 493 bp, Stemona sessilifolia (specimen Hu & Yung 606)
  5S rDNA Spacer clone b
gtgcttgggcgagagtagtactaggatgggtgacctcctgggaagtcctcgtgttgcaccccttctccttttttcgcacgggaatattaagttttgcagaaaaatacagagcggttggcgaaaacaagaatccgtcgtcgcggacgcgttagggatggaatttgatgaaattctcgtctccggccgcctgcggtcgaagtccacgtcgtaggtctctggatgggctttccgtcgggaggtaattcgtccgaaacggacacacgtagcccccgcgatgccgattttcgaggcgtcgcgcaatcgtcgccaaattctgccagcgtgccgtgcctctcctattattactatactatatatccttttttaaaaaaaaaaaaatattatggcctctcccagccagtcgggcgtcattgatggaatcgggagtgcgataggacagggctaagatggaaacgcggtagaaagatgtcgggtgcgatcataccagcactaa

SEQ ID NO: 31 - DNA, 492 bp, Stemona sessilifolia (specimen Zang200401)
  5S rDNA Spacer clone a
gtgcttgggcgagagtagtactaggatgggtgacctcctgggaagtcctcgtgttgcaccccttctccttttttcgcacgggaatattaagttttgcagaaaaatacagagcggttggcgaaaacaagaatccgtcgtcgcagacgcgttagagatggaatttgatgaaattctcgtctccggccgcctgcggtcgaagtccacgtcgtaggtctctggatgggctttccgtcgggaggtaattcgtccgaaacggacaaccgtagcccccgcgacgccgattttcgaggcgtcgcgcaatcgtcgccaaatttcgcaagcgtgccgtgcctatcctattattactatactatatatccttttttttttaaaaaaaaaattatggcctctcccagccagtcgggcgtcgttgatggaatcgggagtgcgataggacggggtcaagatggaaacgcggtagaaagatgtcgggtgcgatcataccagcactaa

SEQ ID NO: 32 - DNA, 493 bp, Stemona sessilifolia (specimen Zang200401)
  5S rDNA Spacer clone b
gtgcttgggcgagagtagtactaggatgggtgacctcctgggaagtcctcgtgttgcaccccttctccttttttcgcacaggaatattaagttttgcagaaaaatacagagcggttggcgaaaacaagaatccgtcgtcgcggacgcgttagagatggaatttgagaaaaattctcgtctccggccgcctgcggtcgaagtccacgtcgtaggtctctggatgggctttccgtcgggaggtaattcgtccgaaacggacacccgtagcccccgcgacgccgattttcgaggcgtcgcgcaatcgtcgccaaattccgccagcgtgccgtgcctatcctattattactatactatatatcctttttttaaaaaaaaaaaaattatggcctctcccagccagtcgggcgtcgttgatggaatcgggagtgcgataggatagggttaagatggaaacgcggtagaaagatgtcgggtgcgatcataccagcactaa

SEQ ID NO: 33 - DNA, 467 bp, Stemona tuberosa (specimen CHAN200401)
  5S rDNA Spacer clone a
gtgcttgggcgagagtagtactaggatgggtgacctcctgggaagtcctcgtgttgcaccccttatctttttggggcacgcggataataagttttggaggaaaccgcggagggcacggccgaaaaaacacgaatccgtcgattcggacgtgttagagttggaatttgaggacgtagtgatccccgacggcctccggctgtcgggcacgccgtaggactcccgatcggctttccgtggacaggtaattcgtccgaaacggacgcccgtagcccccgcggcgacgattttcgtggcgtcgcgcaattcggcactaaacagcaagcacgtagcgtgcatttttttttcttacgggatttgtggcccatttcggcctgtcgagcgtcgctgacggaatcgggagtgcgggacggcggggtcaagaagtaaaagcggaagaaggatgtcgggtgcgatcataccagcactaa

SEQ ID NO: 34 - DNA, 467 bp, Stemona tuberosa (specimen CHAN200401)
  5S rDNA Spacer clone b
gtgcttgggcgagagtagtactaggatgggtgacctcctgggaagtcctcgtgttgcaccccttatctttttggggcacgcggataataagttttggaggaaaccgcggagagacggccgaaaaaacacgaatccgtcgattcggacgtgttagagttggaatttgaggacgtagtgatccccgacggcctccggctgtcgggcacgccgtaggactcccgatcggctttccgtggacaggtaattcgtccgaaacggacgcccgtagcccccgcggcgacgattttcgtggcgtcgcgcaattcggcaccaaacagcaagcacgtagcgtgcattttttttttcttacgggatttgtggcccatttcggcctgtcgagcgtcgctgacggaatcgggagtgcgggacggcggggtcaagaagtaaaagcggaagaaggatgtcgggtgcgatcataccagcactaa

SEQ ID NO: 35 - DNA, 465 bp, Stemona tuberosa (specimen Hu & But 23960)
  5S rDNA Spacer clone a
gtgcttgggcgagagtagtactaggatgggtgacctcctgggaagtcctcgtgttgcaccccttatctttttgcggctcgcggataataagttttggaggaaacgcggagagacggccgaaaaaacacgaatccgtctattcggacgtgttagagttggaatttgaggacgtagtcatccccgacggcctttggccgtcgagcacgccgtaggtctcccgatcggctttccgtggacaggtaattcgtccgaaacggacgcccgtagcccccgcggcgacgattttcgtggcgtcgcgcaattcggcacctaacagcaagcacgtagcgtgcatttttttttcttacgggatttatggcccacttcggccagtcgagcgtcgctgacggaatcgggagtgcgggacggcggggctgagaagtaaaagcggacgaaagatgtcgggtgcgatcataccagcactaa

SEQ ID NO: 36 - DNA, 467 bp, Stemona tuberosa (specimen Hu & But 23960)
  5S rDNA Spacer clone b
gtgcttgggcgagagtagtactaggatgggtgacctcctgggaagtcctcgtgttgcaccccttatctttttgcggctcgcggataataagttttggaggaaaccgcggagagacggccgaaaaaacacgaatccgtctattcggacgtgttagagttggaatttgaggacgtagtcatccccgacggcctttggccgtcgagcacgccgtaggtctcccgatcggctttccgtggacaggtaattcgtccgaaacggacgcccgtagcccccgcggcgacgattttcgtggcgtcgcgcaattcggcacctaacagcaagcacgtagcgtgcattttttttttcttacgggatttatggcccacttcggccagtcgagcgtcgctgacggaatcgggagtgcgggacggcggggctgagaagtaaaagcggacgaaagatgtcgggtgcgatcataccagcactaa

SEQ ID NO: 37 - DNA, 468 bp, Stemona tuberosa (specimen ICM20042540)
  5S rDNA Spacer clone a
gtgcttgggcgagagtagtactaggatgggtgacctcctgggaagtcctcgtgttgcaccccttatctttttggggcacgcggataataagttttggaggaaaccgcggggagacggccgaaaaaacacgaatccgtcgattcggacgtgttagagttggaatttgaggacgtagtcgtccccgacggccatcggccgtcgggcacgccgtaggtctcccgatcggctttccgtggacaggtaattcgtccgaaacggacgcccgtagcccccgcggcgacgattttcgtggcgtcgcgcaattcggcaccaaacagcaagcacgtagcgtgcatttttttttttcttacgggatttatggcccacttcggccagtcgagcgtcgctgacggaatcgggagtgcgggacggcagggctaagaattaaaagcggacgaaagatgtcgggtgcgatcataccagcactaa

SEQ ID NO: 38 - DNA, 468 bp, Stemona tuberosa (specimen ICM20042540)
  5S rDNA Spacer clone b
gtgcttgggcgagagtagtactaggatgggtgacctcctgggaagtcgtcgtgttgcaccccttatctttttggggcacgcggataataagttttggaggaaaccgcggggagacggccgaaaaaacacgaatctgtcgattcggacgtgttagagttggaatttgaggacgtagtcgtccccgacggccatcggcggtcaggcacgccgtaggtctcccgatcggctttcggtggacaggtaattcgtccgaaacggacgcccgtagtccccgcagcgacgattttcgtggcgtcgcgtaattcgacaccaaacagcaagcacgtagcgtgcatttttttttttcttacgggatttatggcccacttcggccagtcgagcgtcgctgacggaatcgggagtgcggggcggcagggctaagaagtaaaagcggcagaaagatgtcgggtgcgatcataccagcactaa

SEQ ID NO: 39 - DNA, 467 bp, Stemona tuberosa (specimen ICM20042540)
  5S rDNA Spacer clone c
gtgcttgggcgagagtagtactaggatgggtgacctcctgggaagtcctcgtgttgcaccccttatctttttggggcacgcggataataagttttggaggaaaccgcggagagacggccgaaaaaacacgaatccgtcgattcggacgtgttagagttggaatttgaggacgtagtcgtccccgacggccatcggcggtcgggcatgccgtaggtctcccgatcggctttccgtggacaggtaattcgtccgaaacggacgcccgtagcccccgcggcgacgattttcgtggcgtcgagcaattcggcaccaaacagcaagcacgtagcgtgcattttttttttcttacaggatttatggcccacttcggccagtcgagcgtcgctgacggaatcgggagtgcgggacggcagggctaagaagtaaaagcggcagaaagatgtcgggtgcgatcataccagcactaa

SEQ ID NO: 40 - DNA, 467 bp, Stemona tuberosa (specimen ICM20042541)
  5S rDNA Spacer clone a
gtgcttgggcgagagtagtactaggatgggtgacctcctgggaagtcctcgtgttgcaccccttttctttttggggcacgcggataatgagttttggaggaaaccgcagagagacggccgaaaaaacacgaatccgtcgattcggacgtgttagagttggaatttgaggaagtagtcatccccgacggcccccggcggtcggccccgtcgtacgtctccggatcggctttccgtggacaggtaattcgtccgaaacggacacccgtagcccccgcggcgtcgattttcgtggcgtcgcgcaattcggcaacaaattgcaagcacgcagcgtggttttctacttttttatgggatttatggccaactccggccactcgagcgtcgttgacggaatcgggagcgcgggactgcggggttaagaagtaaaagcggaagaaggttgtcgggtgcgatcataccagcactaa

SEQ ID NO: 41 - DNA, 467 bp, Stemona tuberosa (specimen ICM20042541)
  5S rDNA Spacer clone b
gtgcttgggcgagagtagtactaggatgggtgacctcctgggaagtcctcgtgttgcaccccttttctttttggggcacgcggataatgagttttggaggaaaccgcagagagacggccgaaaaaacacgaatccgtcgattcggacgtgttagagttggaatttgaggaagtagtcatccccgacggcctccggctgtcggccccgtcgtacgtctccggatcggctttccgtggacaggtaattcgtccgaaacggacacccgtagcccccgcagcgtcgattttcgtggcgtcgcgcaattcggcaacaaattgcaagcacgcagcgtggttttctacgtttttatgggatttatggccaactccggccactcgagcgtcgttgacggaaacgggagcgcgggactgcggggttaagaagtaaaagcggaagaaggttgtcgggtgcgatcataccagcactaa

SEQ ID NO: 42 - DNA, 466 bp, Stemona tuberosa (specimen Woo 23973)
  5S rDNA Spacer clone a
gtgcttgggcgagagtagtactaggatgggtgacctcctgggaagtcctcgtgttgcaccccttatctttttggtgcacgcggataataagttttggaggaaaccgcggagagacggccgaaaaatcacgaatccgtcgattcggacgtgttagagttggaatttgaggaagcagtcgtcgccgacggccgccggctgtcggccacgtcgtaggtctcccgatcggctttccgtggacaggtaattcgtccgaaacggacacccgtagcccccgcggcgacgattttcgtggcgtcgcgcaattcggcaccaaacagcaagcacgtagcgtgcattttttttttttatgggatttatggcccacttcggccactcgagcgtcgctgacggaatcgggagtgcgggacagcgggtttatgaagtaaaagcggaagaaagatgtcgggtgcgatcataccagcactaa

SEQ ID NO: 43 - DNA, 462 bp, Stemona tuberosa (specimen Woo 23973)
  5S rDNA Spacer clone b
gtgcttgggcgagagtagtactaggatgggtgacctcctgggaagtcctcgtgttgcaccccttatctttttggtgcacgtggataataagttttggaggaaaccgcggagagacggccgaaaaatcacgaatccgtcgattcggacgtgttagagttggaatttgaggaagtagtcatcgccgacggcctttggctgtcggccacatcgtaggtctcccgatcggcttttcgtggacaggtaattcgtccgaaacggacacccgcagcccccgcggcaaagattttcgtggcgtcgcgcaattcggcaccaaacagcaagcacgtagcgtgcattttttttttgggatttatggcccacttcggccactcgagcgtcgctgacgtaatcgggagtgcgggacagcgggtttatgaagtaaaagcggaagaaagatgttgggtgcgatcataccagcactaa

SEQ ID NO: 44 - DNA, 465 bp, Stemona tuberosa (specimen Woo 23973)
  5S rDNA Spacer clone c
gtgcttgggcgagagtagtactaggatgggtgacgctcctgggaagtcctcgtgttgcaccccttatctttttggtgcacgcggataatatgttttggaggaaaccgcggagagacggtcgaaaaatcacgagtccgtcgattcggacgtgctagagtcagaatttgaggaagtagtcgtcgcggacggccttcggctgtcggccacgtcgtaggtctcccgatcggctttccgtggacaggtaattcgtccgaaacggactcccgtagcacccgcggcgtcgattttcgtggcgtcgcgcaattcggcaccaaagagcaagcacgtggcgtgcattttttttttatgggatttctggcccacttcggccactcgagcgtcgctgaaggaatcgggagtgcgggacagcgggtttaagaagtaaaagcggaagaaagatgtcgggtgcgatcataccagcactaa

SEQ ID NO: 45 - DNA, 461 bp, Stemona tuberosa (specimen Woo 23973)
  5S rDNA Spacer clone d
gtgcttgggcgagagtagtactaggatgggtgacctcctgggaagtcctcgtgttgcaccccttatctttttggtgcacgcggataataagttttggaggaaaccgcggagagacggccgacaaatcacgaatccatcgattcggacgtgttagagttggaatttgaggaagtagtcgtcgccgacggccgttggctgtcggccacgtcgtaggtctcccgatcggcttttcgtggacaggtaattcgtccgaaacggacacccgcagcccccgcggcaaagattttcgtggcgtcgcgcaattcggcaccaaacagcaagcacgtagcgtgcatttttttttatgggatttatggcccacttcggccactcgagcgtcgctgacgtaatcgggagtgcgggacagcgggtttatgaagtaaaagcggaagaaagatgtcgggtgcgatcaccagcactaa

SEQ ID NO: 46 - DNA, 461 bp, Stemona tuberosa (specimen Woo 23973); identical to SEQ ID NO: 45
  5S rDNA Spacer clone e

SEQ ID NO: 47 - DNA, 466 bp, Stemona tuberosa (specimen Woo 23973); identical to SEQ ID NO: 42
  5S rDNA Spacer clone f

(19) United States
(12) Patent Application Publication
(10) Pub. No.: US 2007/0015192 A1
(43) Pub. Date: Jan. 18, 2007

(54) Method of herbal authentication based on 5S rRNA spacer sequences

(75) Inventors: Paul Pui-Hay But, Tai Po (HK); Pang-Chui Shaw, New Territories (HK); Ren-Wang Jiang, New Territories (HK); Yiu-Man Chan, Tai Kok Tsui (HK)

Correspondence Address: FISH & RICHARDSON PC, P.O. BOX 1022, MINNEAPOLIS, MN, 55440-1022, US

(73) Assignee: HONG KONG JOCKEY CLUB INSTITUTE OF CHINESE MEDICINE LIMITED

(21) Appl. No.: 11/479,121

(22) Filed: Jun. 30, 2006

Related U.S. Application Data
(60) Provisional application No. 60/697,573, filed on Jul. 11, 2005.

(51) Int. Cl. C12Q 1/68

(52) U.S. Cl. 435/6

(57) ABSTRACT

A method for authenticating herbs or plants based on sequences in trnL and/or 5S rRNA coding region. Using this method, the sequences from samples to be authenticated and samples of known identity herbs or plants are compared based on similarity percentage calculation. The authentication may also be based on phylogenetic trees or parsimonious trees generated based on DNA sequence analyses.

### CROSS REFERENCE TO RELATED APPLICATION

This application claims priority to U.S. Provisional Application Serial No. 60/697,573, filed Jul. 11, 2005, the contents of which are hereby incorporated by reference.

### FIELD OF THE INVENTION

This invention relates to a method for authenticating herbs or plants by analyzing DNA sequences. Particularly, it relates to an herb authentication method based on genomic DNA sequences in the 5S rRNA spacer.

### BACKGROUND OF THE INVENTION

Natural resources, such as botanical products (herbs), animal products and minerals have long been used in many parts of the world for health and medical purposes, treating or preventing diseases. Nowadays, even with the advance of modern medicine, the market for those traditional medicinal materials remains prosperous in many countries. Some people used them as a main treatment measure, for example, in many Asian countries, while others use them as an alternative treatment measure.

Among traditional medicinal materials, botanical products (or herbs) are the main components. Herbs are normally processed parts of various plants, such as roots, stems, leaves, flowers, fruits, seeds, etc. From the very beginning, herb authentication has presented a great challenge for people using them for medical purposes. When wrong herbs are used, they may be ineffective and thus delay the treatment of the diseases, or they may worsen the conditions and may even cause death. Misidentification of herbs can be non-intentional (processed plant parts are inherently difficult to distinguish) or intentional (profit-driven merchants sometimes substitute expensive herbs with less-expensive look-alike ones). Traditionally, people authenticated herbs by looking at their appearance, smelling them and/or tasting them, and some of those methods are still being used. Later, herbs are authenticated by inspection under microscopes, where the shape and content of various plant cells are examined and analyzed. Those methods based on organoleptic markers or anatomical characters are sometimes indeterminate. Analytical chromatography, such as thin-layer chromatography, high-performance liquid chromatography, or liquid chromatography/mass spectrometry are being used for herb authentication. However, chemical profiles are variable and can be affected by factors other than authenticity. More recently, with the advance of modern biotechnology, genomic DNA fragments in certain regions are amplified by polymerase chain reaction (PCR) and subject to restriction enzyme digestion, whereby producing restriction digestion profiles, which are in turn used for ascertaining the authenticity of certain particular herbs based on comparison with the profiles of a standard (sample of known identity) (see U.S. Pat. Nos. 5,876977, 6,309,840 and 6,803,215).

However, even those modern authentication techniques cannot be universally applicable to all herbal species. There remains the need for developing reliable authentication methods on a species-by-species basis.

### SUMMARY OF THE INVENTION

The present invention provides a new technique to authenticate herbs or plants from the plant genus Stemona. Three species of this genus, Stemona japonica, S. sessilifolia and S. tuberosa, are officially recognized in China as the source for the herb Radix Stemonae (dried root tubers of the plants). The authentication method of the present invention is based on analysis of the DNA sequences of the 5S rDNA spacer regions. Using these regions as molecular markers, the technique of the present invention provides a reliable method for authenticating Radix Stemonae, an otherwise very challenging task because dried root tubers of different Stemona species and adulterants are very similar in appearance and their chemical profiles are quite variable. As one aspect of the invention, there is provided a repeatable and objective method for authenticating Radix Stemonae. The method is simple and can be practiced in any standard molecular biology laboratories.

As a particular embodiment of prevent invention, 17 samples representing six Stemona species and one Asparagus species were subjected to the authentication method of the present invention. In general, the authentication process involves some or all of the following steps:

(a) Extracting DNA from the plant material samples (either fresh or dried plants);

(b) Amplifying DNA extracts by the PCR (polymerase chain reaction) technology with suitable primers;

(c) Inserting the PCR products into vector and then transform it into competent cells, culturing competent cells clones individually and extracting the plasmid DNA.

(d) Sequencing amplified plasmid DNA of individual clones;

(d) comparing and aligning sequences of different samples and/or from a standard sequence; and

(f) calculating the percentage similarity among the samples themselves or with the standard sequence.

A conclusion of authenticity can be drawn based on the obtained percentage similarity for each sample, or based on phylogenetic trees or parsimonious trees generated following various DNA sequence analysis. As one object of the present invention, there is provided a plurality of novel DNA sequences as the standard sequence for practicing the authentication method of the present invention.

The following DNA sequences are provided standards used for practicing the present invention. Each of the listed sequences can be used in its entirety as a standard sequence for comparison. Or a portion of a listed sequence, from a 10% portion to 100% portion (which equals to entire sequence), can be used as a standard sequence to practice the present invention. In the specific embodiment described below, the 100% portion of the listed sequence is used as the standard sequence. People of ordinary skill in the art can readily use a lesser portion of the provided standard sequences and can determine the optimal size of the portion for a particular authentication task. Of course, it is contemplated by the present invention that a larger DNA sequence that encompasses one of the listed sequences may also be used as the standard sequence to practice the present invention by using different primers.

### BRIEF DESCRIPTION OF THE DRAWINGS

FIG. 1A-1N show sequence alignment of the 5S rRNA spacers of Stemona species and Asparagus filicinus.

FIG. 2 shows phylogenetic tree generated by UPGMA tree construction method based on 5S rRNA spacer sequences.

FIG. 3 shows phylogenetic tree generated by Neighbour-joining analysis based on 5S rRNA spacer sequences.

FIG. 4 shows strict consensus parsimonious tree generated based on 5S rRNA spacer sequence.

### DETAILED DESCRIPTION OF THE PRESENT INVENTION

Herb Samples

17 samples representing six Stemona species and one Asparagus species were collected. The 17 samples are either fresh plants or dried plant materials. The samples and their sources are provided as illustration only (Table 1), and as an authentication method, the source of the sample is unimportant as long as there is a known standard sample available that has been authenticated.

TABLE 1
The sourses and label of 17 samples representing six Stemona species
and one Asparagus species.

| | | Materials (with names in Chinese | |
|---|---|---|---|
| | Label | characters) | Sources |
| 1. | ICM 20042543 | Stemona japonica [symbol] | Institute of Medicinal Plant |
| | | | Development, Chinese |
| | | | Academy of Medical Sciences, |
| | | | Beijing |
| 2. | Hu and But | Stemona japonica [symbol] | Anhui Province |
| | 24032 | | |
| 3. | Hu & But | Stemona japonica [symbol] | Nanjing Institute of |
| | 23971 | | Botany, Chinese Academy of |
| | | | Sciences |
| 4. | Ma 9066 | Stemona parviflora [symbol] | Hainan Province |
| 5. | Hu and But | Stemona parviflora [symbol] | Hainan Province |
| | 24034 | | |
| 6. | HU & But | Stemona sessilifolia [symbol] | Institute of Medicinal Plant |
| | 23972 | | Development, Chinese |
| | | | Academy of Medical |
| | | | Sciences, Beijing |
| 7. | Hu & Yung | Stemona sessilifolia [symbol] | China Phamaceutical |
| | 606 | | University |
| 8. | Zang 200401 | Stemona sessilifolia [symbol] | Shandong Province |
| | | (=Stemona shandongensis [symbol] ) | |
| 9. | Chan 200401 | Stemona tuberosa [symbol] | South China Institute of |
| | | | Botany, Academy of |
| | | | Sciences |
| 10. | Woo 23973 | Stemona tuberosa [symbol] | Guang Xi Province |
| 11. | ICM | Stemona tuberosa [symbol] | Yunan Province |
| | 20042541 | | |
| 12. | Hu & But | Stemona tuberosa [symbol] | Hong Kong |
| | 23960 | | |
| 13. | ICM | Stemona tuberosa [symbol] | Purchased from Tong ren |
| | 20042540 | | tong, Beijing (originally |
| | | | cultivated in Guang Dong) |
| 14. | ICM | Asparagus filicinus [symbol] | Yunnan Province |
| | 20042542 | | |
| 15. | SK15 | Stemona kerrii [symbol] | Thailand |
| 16. | SK 32 | Stemona kerrii [symbol] | Thailand |
| 17. | G57 | Stemona collinsae [symbol] | Thailand |

DNA Extraction

Before DNA extraction, the plant materials were treated to avoid fungal contamination. Washed plant materials were ground into powder in liquid nitrogen by pestle and mortar. The powders were kept at −80° C. until use. Genomic DNA samples were extracted from plant samples (herbs) by the Kang et. al (1998) method. The powdered sample was mixed with 400 μl of SDS extraction buffer (200 mM Tris-HCI (pH 8.0), 200 mM NaCl, 25 mM EDTA, 0.5% SDS) and 50 μg proteinase K. The mixture was incubated at 37° C. for 1 h. Then, 400 μl of 2% CTAB solution (100 mM Tris-HCI (pH 8.0), 1.4 M NaCl, 20 mM EDTA (pH 8.0), 2% (w/v) CTAB, 1% PVP (polyvinylpyrrolidone) Mr 40000) was added and mixed well. The mixture was then mixed with chloroform:isoamyl alcohol (24:1) with 5% phenol and then centrifuged at 12,000 rpm for 10 min. The supernatant was mixed with ⅔ volume isopropanol and incubated at room temperature for 10 min. At last, the mixture was centrifuged at 12,000 rpm for 5 min. The pellet was washed with 70% ethanol and resuspended in 50 μl autoclaved double distilled water.

Polymerase Chain Reaction (PCR)

The DNA regions of interest were amplified by PCR. The 5S rRNA spacers were amplified using primers S-1 (5′-GGA TCC GTG CTT GGG CGA GAG TAG TA -3′ or SEQ ID NO: 1) and AS-1 (5′-GGA TCC TTA GTG CTG GTA TGA TCG CA -3′ or SEQ ID NO: 2). Another pair of primers 5S2F (5′-GTG CTT GGG CGA GAG TAG TA-3′ or SEQ ID NO: 3) and 5S2R (5′-TTA GTG CTG GTA TGA TCG CA -3′ or SEQ ID NO: 4) was used if PCR was not successful with S-1 and AS-1. The two primer pairs anneal at the same region of the genome but the 5S2F and 5S2R are 6 bp shorter than S-1 and AS-1. The specific primers provided here is by way of example, not limitation. Certainly, other primers may be chosen and provide satisfactory results in amplifying the desired DNA regions.

PCR was performed in a mixture containing 15.3 μl autoclaved double distilled water, 2.5μl 10X PCR buffer (100 mM Tris-HCI (pH 8.8), 500 mM KCI), 2 μl 2.5 mM dNTP, 2 μl 25 mM MgCI₂, 1 u Taq polymerase, 1 μl (10 mM) of both primers and 1 μl template DNA. Thermal cycling was performed in a MJ-PTC100 thermocycler and carried out as follows: one cycle of 95° C. for 5 min; then 20 cycles of 95° C. for 20 sec, 56° C. for 30 sec and 72° C. for 1.5 min; and a final extension at 72° C. for 5 min. The sizes of the 5S rRNA spacer of Stemona species is about 500 bp long. The 5S rRNA spacer in Asparagus filicinus is 600 bp.

Ligation and Transformation

The resulting PCR products are purified and then inserted into vestor through ligation. Ligation of PCR products as performed using pGEM®-T Easy Vector System I (Promega). The resulting recombinant plasmid is transformed into Escherichia coli DH5α competent cells. 100 μl E. coli competent cells was taken out from −80° C. and kept on ice to thaw the cells. When the cells were just thawed, all 5 μl recombinant plasmid was added. The competent cells were left on ice for 30 min and then heat-shocked at 42° C. for 2 min. After heat-shock, the competent cells were kept on ice for further 2 min. Then 200 μl of 37° C. LB medium was added to the cells and the mixture was incubated at 37° C. for 50 min. After incubation, 50 μl 2% X-gal and 5 μl 0.4 M IPTG were added to the cells. The culture were spread on a Luria-Bertani (LB) Agar plate and incubated at 37° C. overnight. After incubation, blue and white colonies were formed on the plate. For each plate, several white colonies were selected. Each single white colony was inoculated into 5 ml Luria-Bertani medium containing 50 μg/ml ampicillin. The cultures were incubated at 37° C. overnight with continuous shaking. Plasmid DNA was isolated from the cultures using Mini-M™ Plasmid DNA Extraction System (Viogene, cat. GF1002).

DNA Sequencing

BigDye® Terminator v3.1 Cycle Sequencing Kit (Applied Biosystems, PN 4337455) was used for cycle sequencing of the plasmid DNA. The products of cycle sequencing were purified and then resuspended in HiDi Formamide (Applied Biosystems, PN 4311320). ABI PRISM® 3100 Genetic Analyzer was then used for electrophoresis of the samples.

DNA Authentication of Radix Stemonae

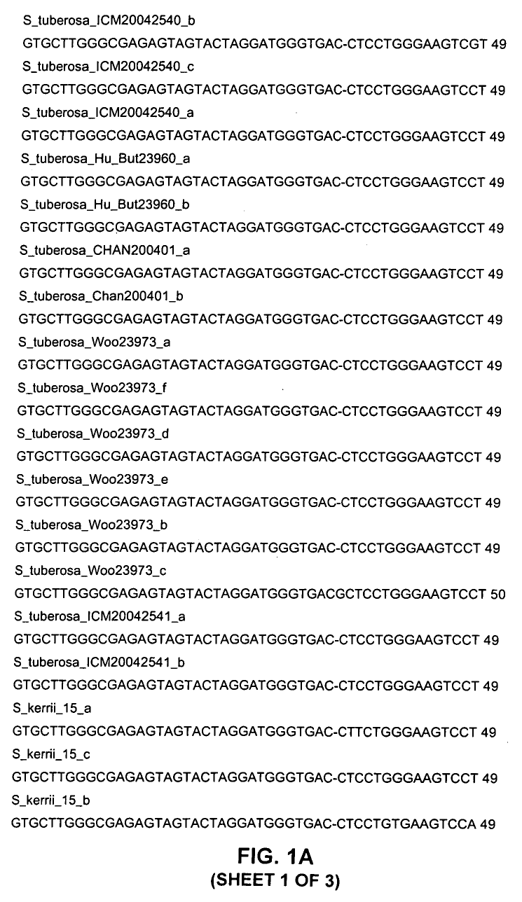

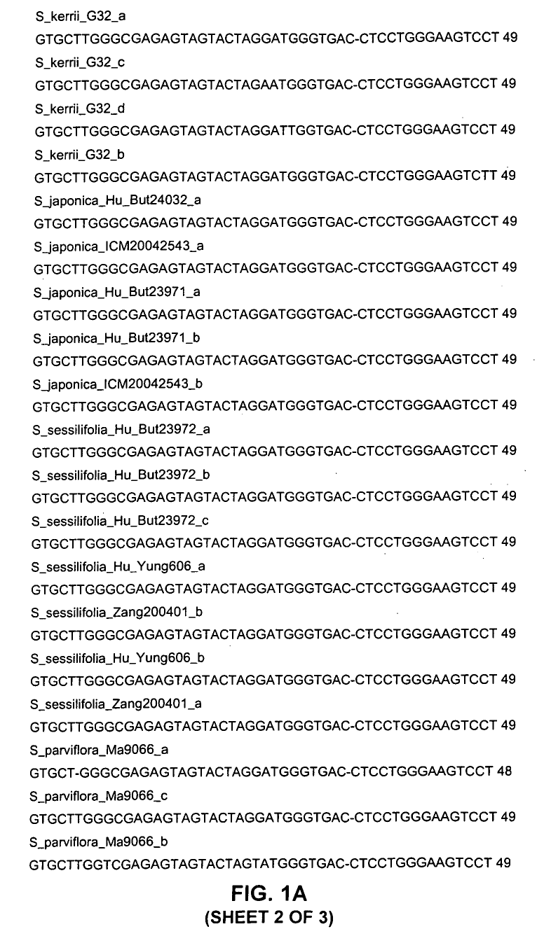

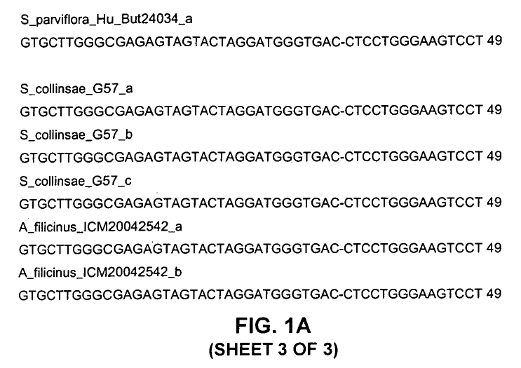

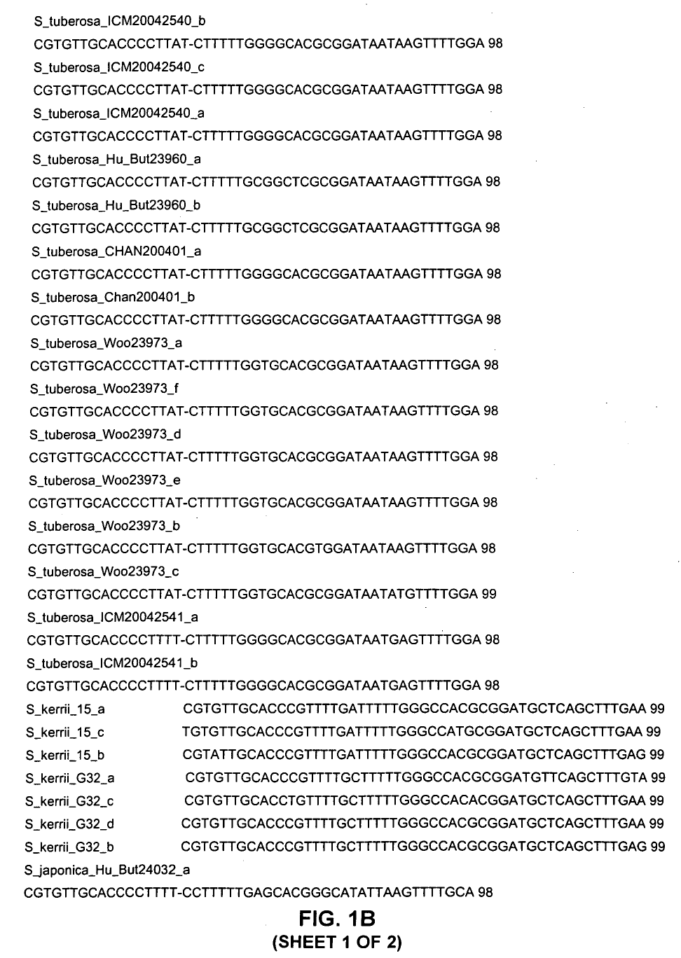

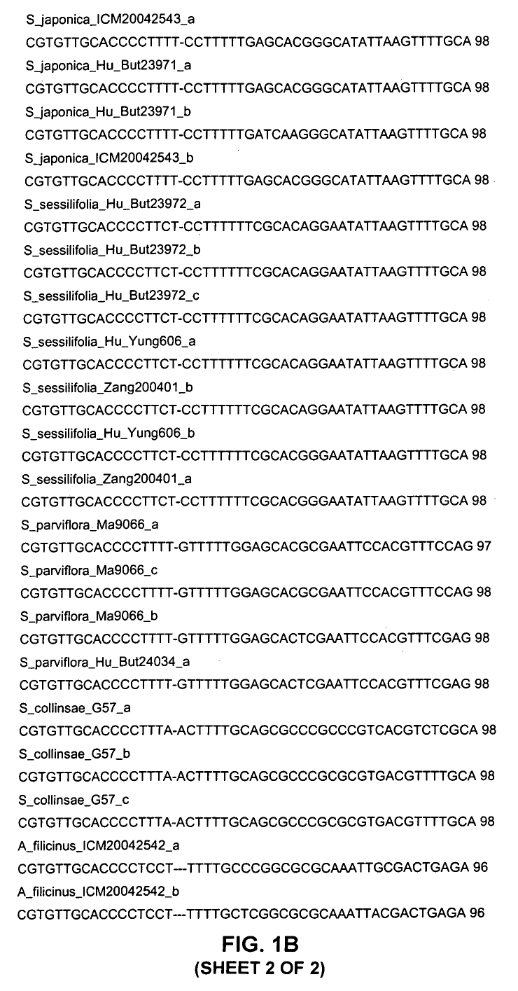

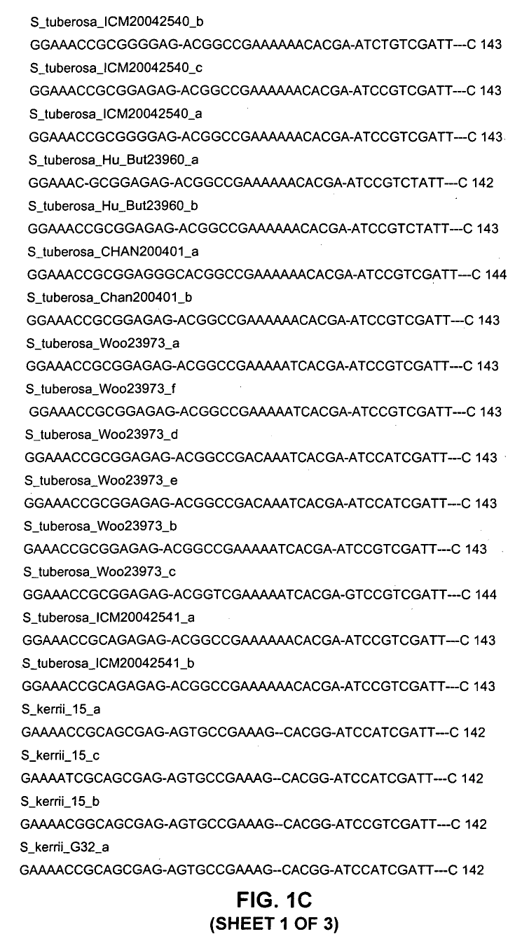

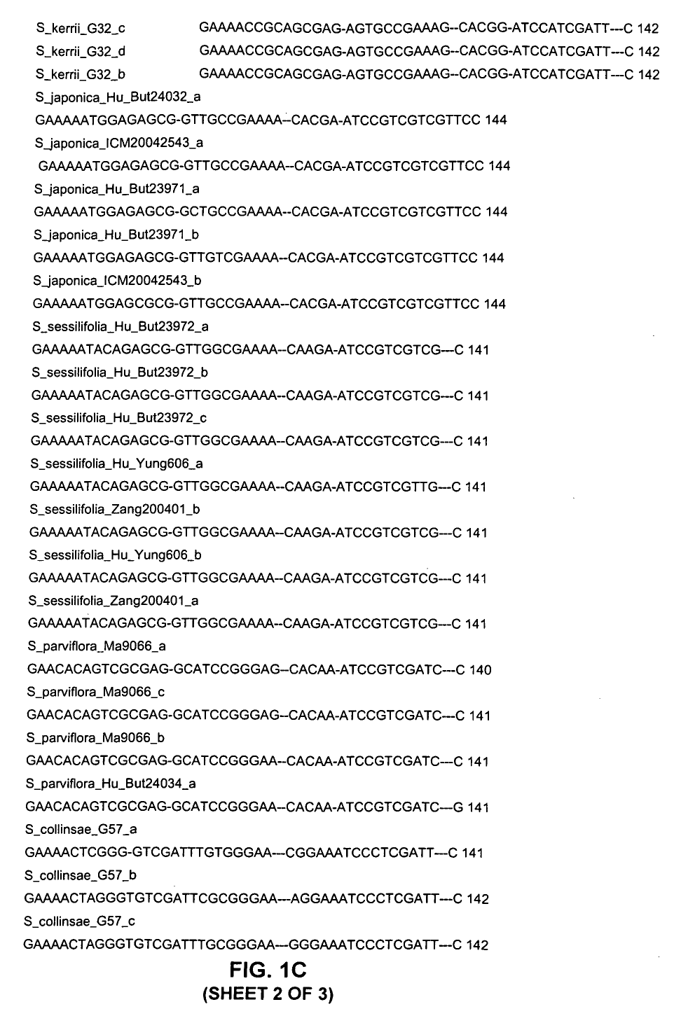

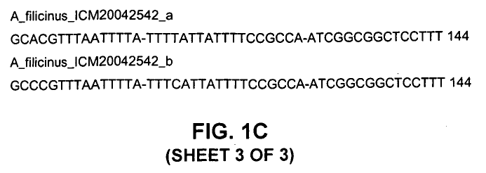

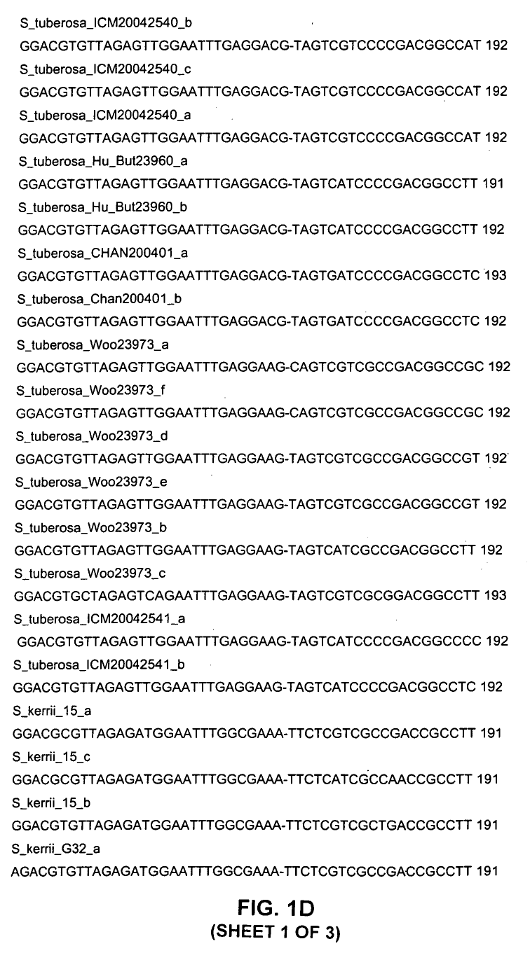

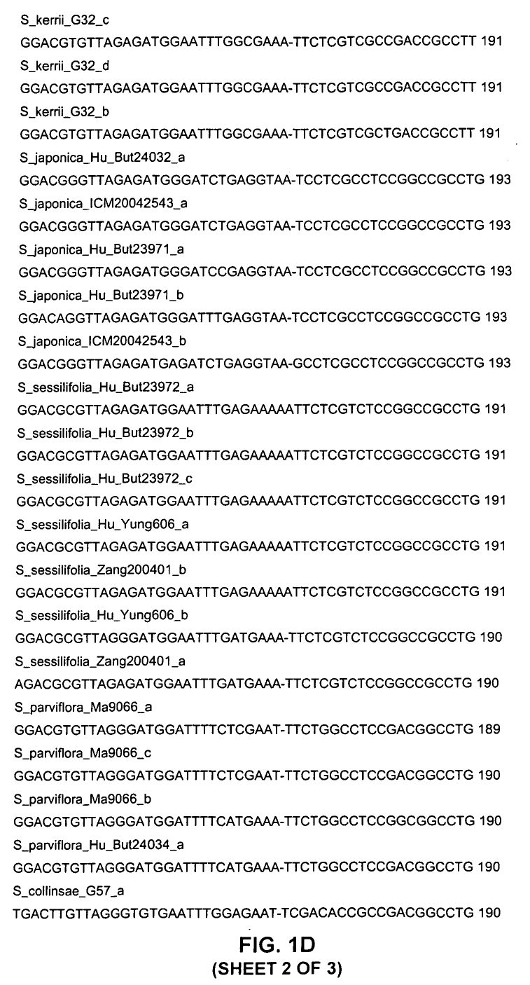

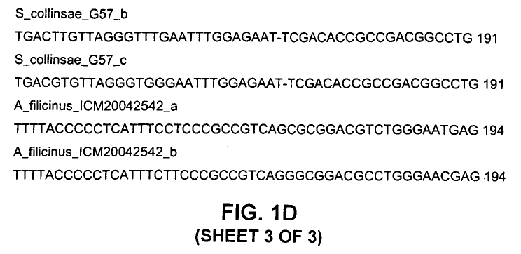

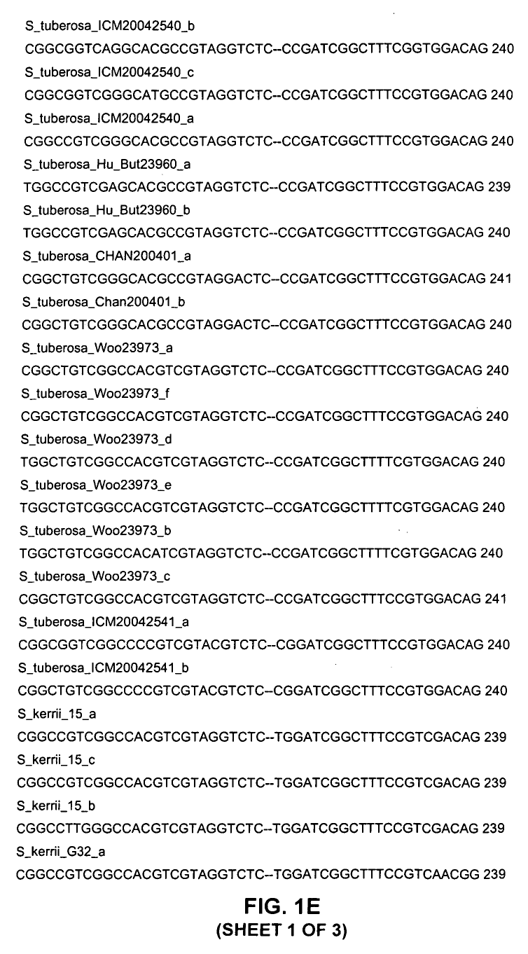

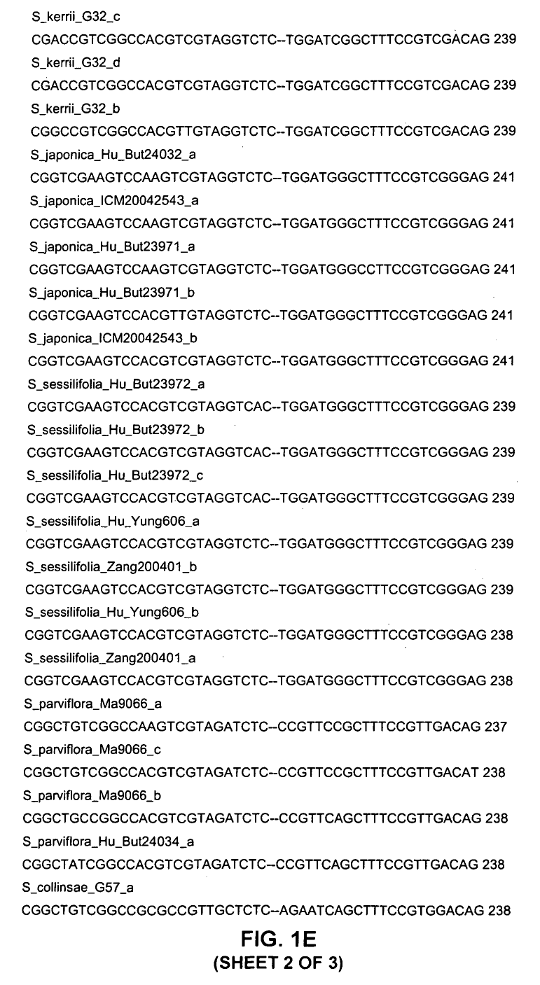

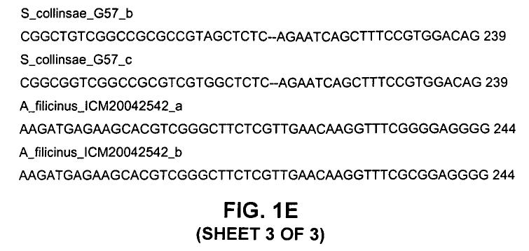

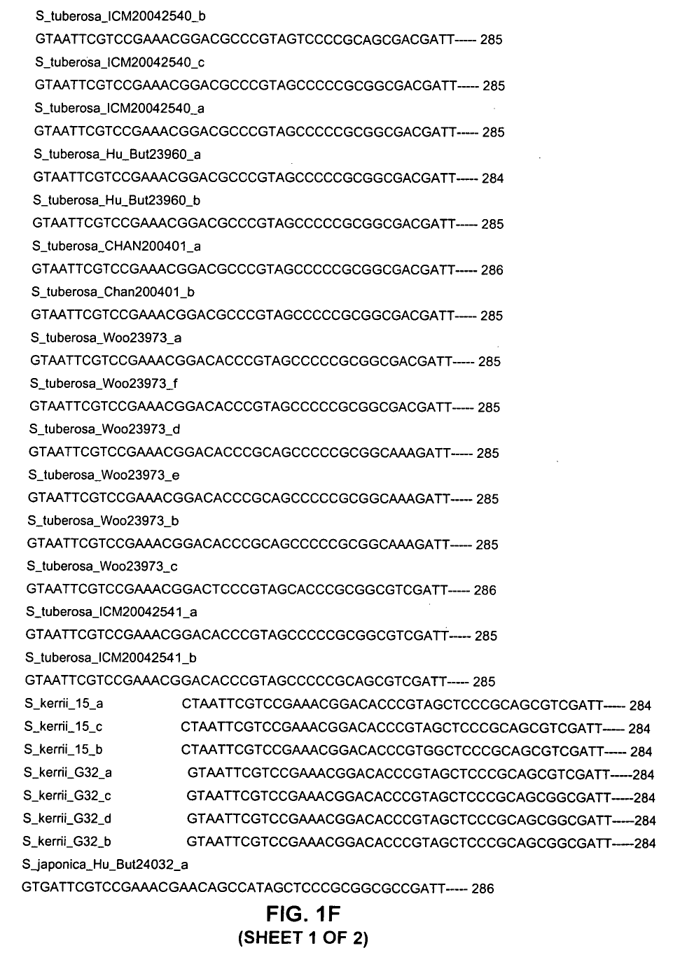

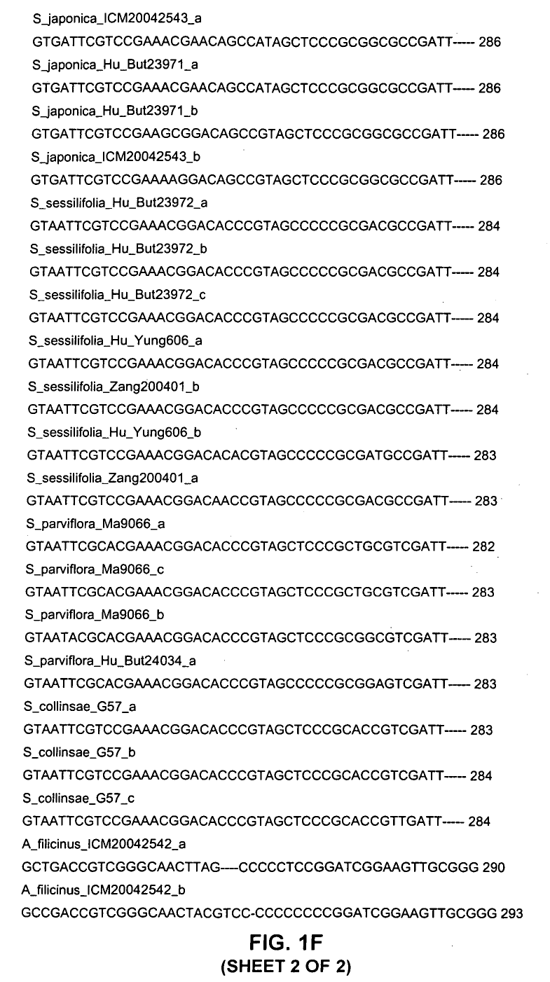

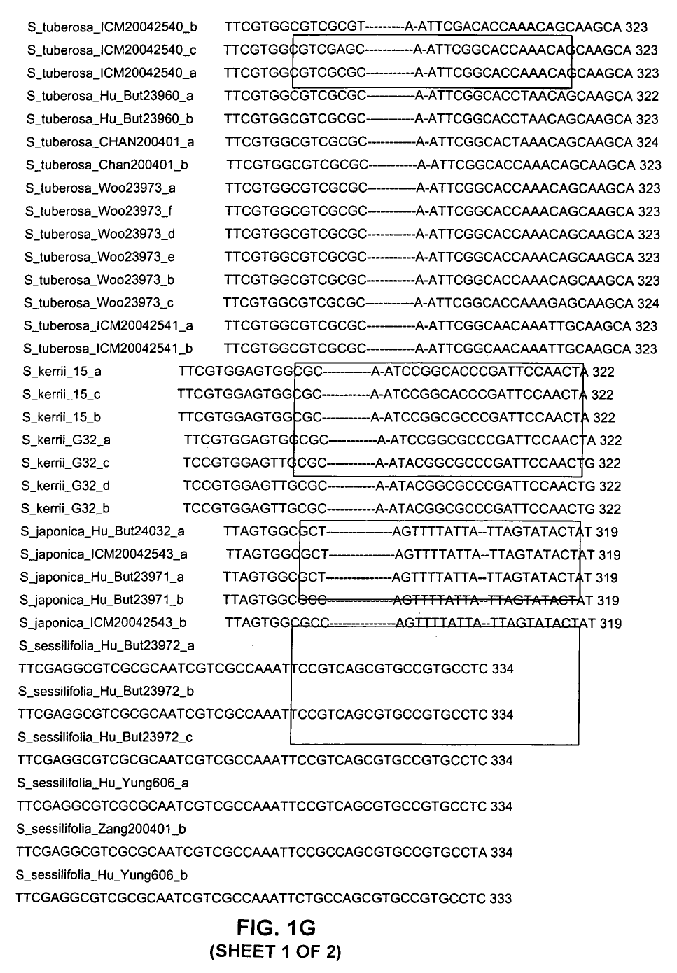

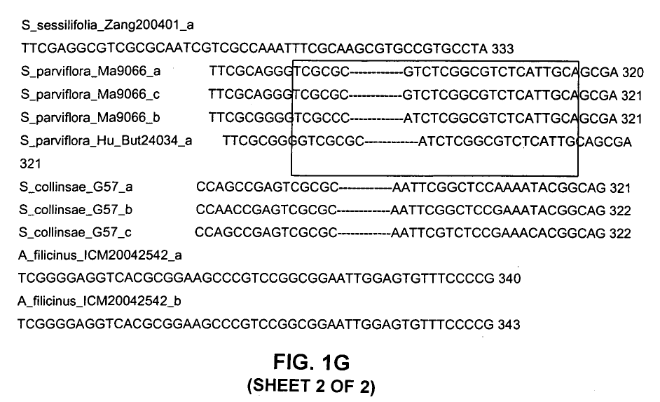

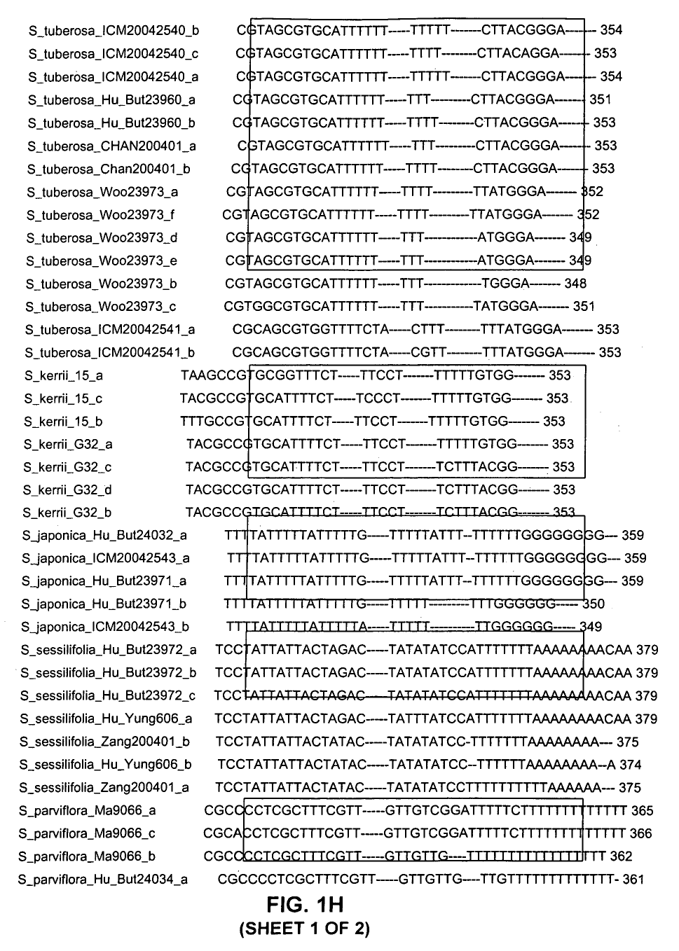

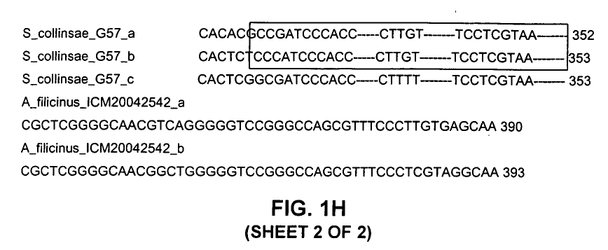

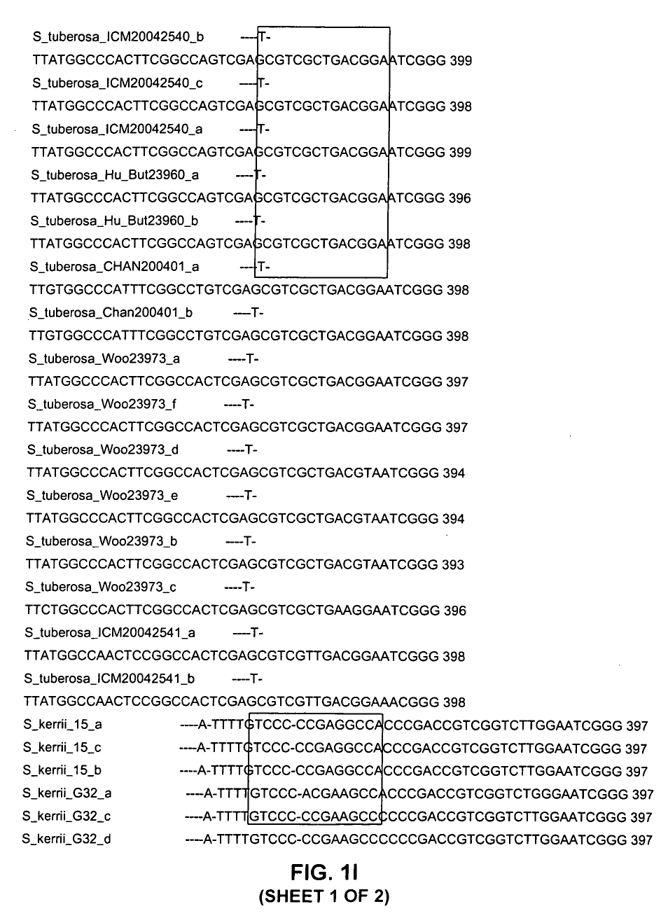

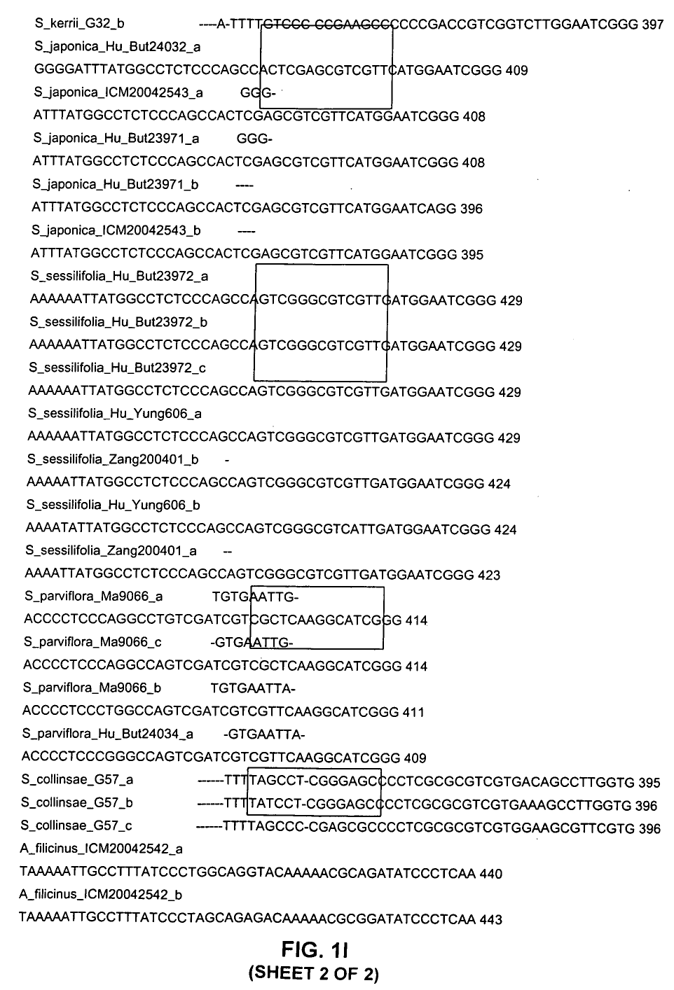

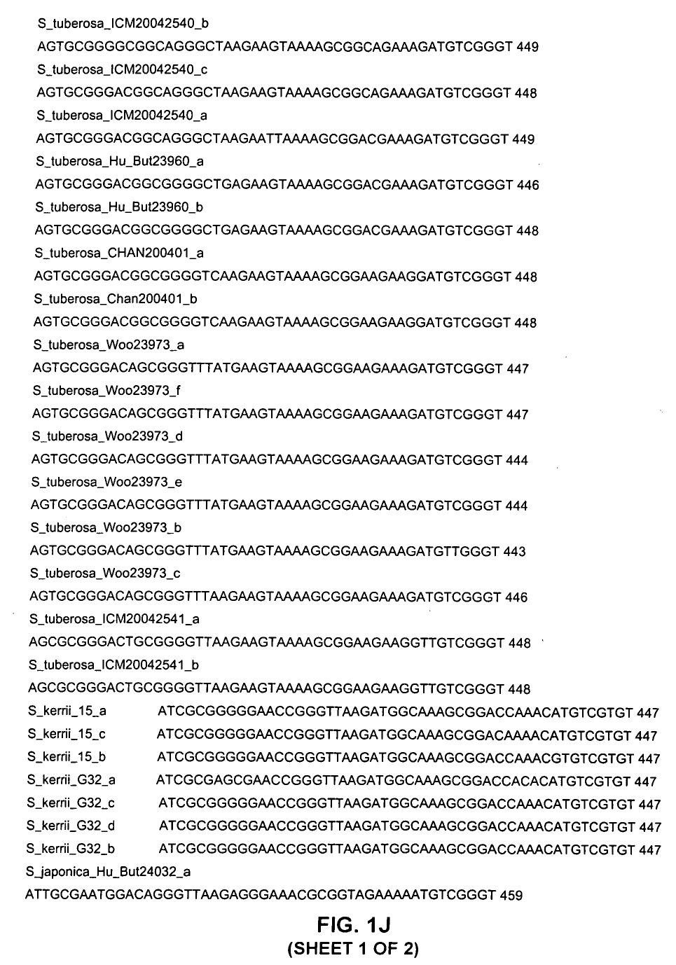

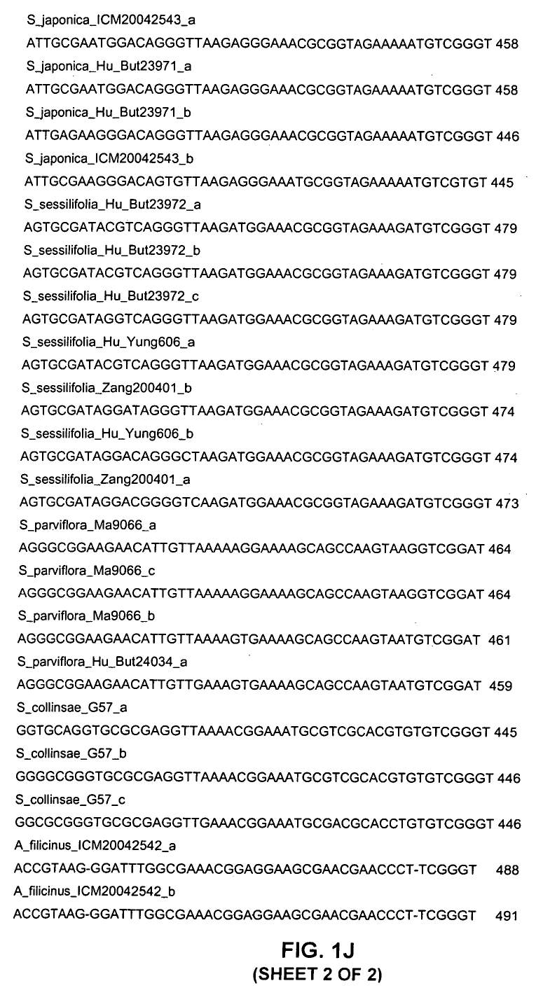

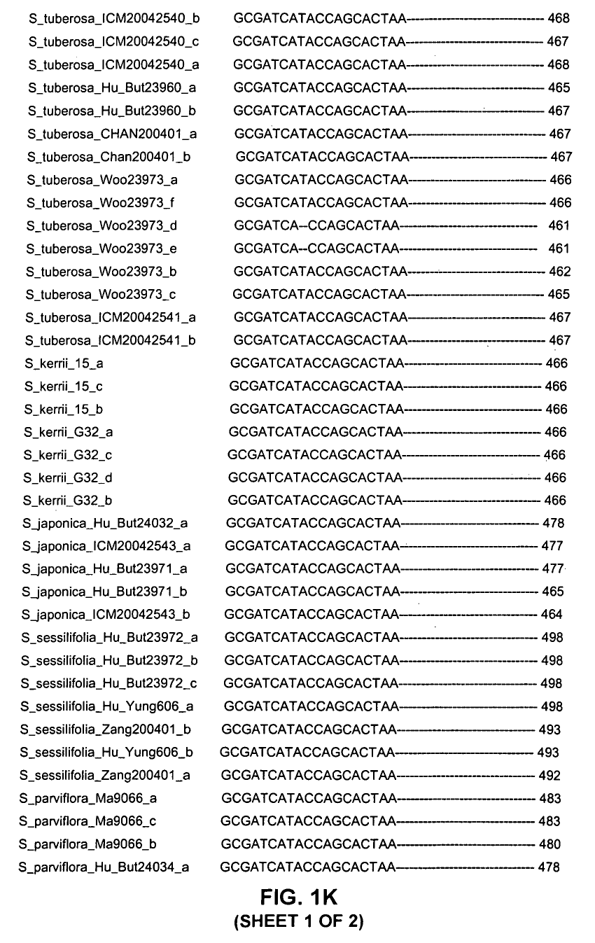

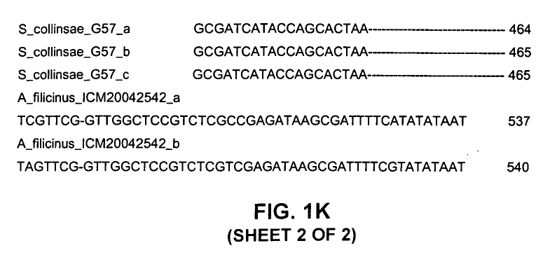

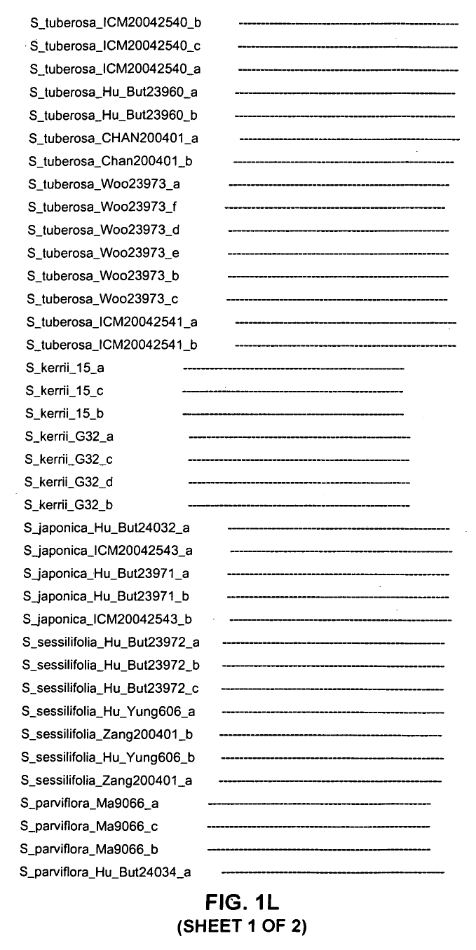

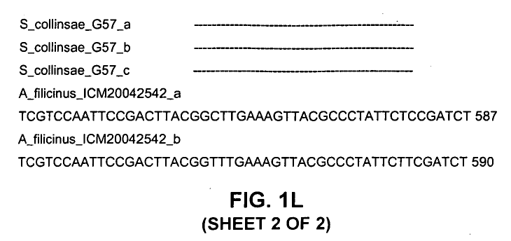

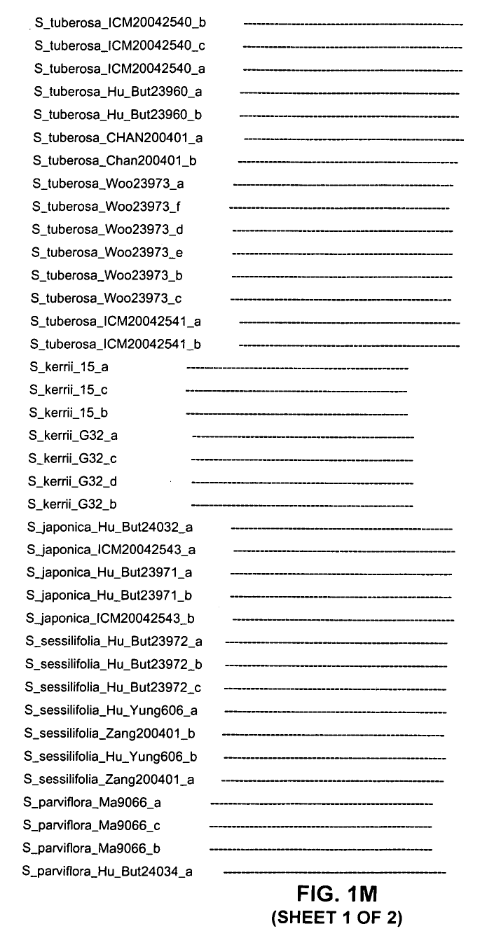

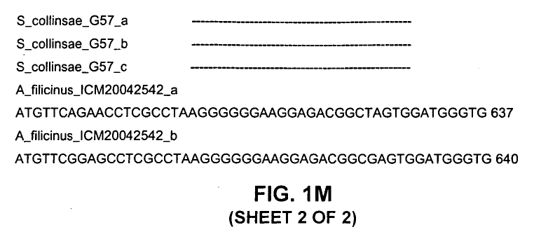

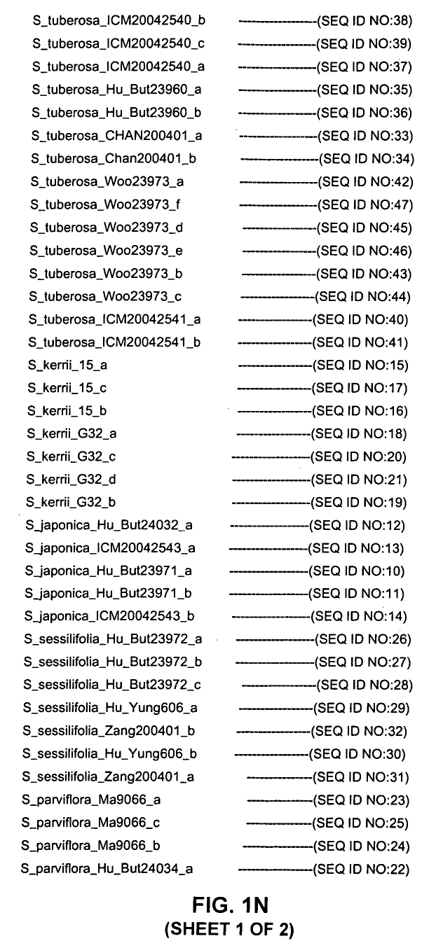

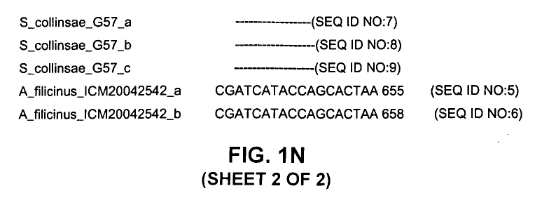

DNA sequences of Stemona collinsae, S. japonica, S. kerrii, S. parviflora, S. sessilifolia, S. tuberosa and Asparagus filicinus were compared. The sequences were aligned and shown in FIG. 1. The percentage similarity among the samples was calculated and the result is presented in Tables 2. Phylogenetic trees were constructed to visualize the relationship. Molecular Evolutionary Genetics Analysis software (MEGA) version 2.1 (Kumar et al. 2001) was used for generating Unweighted Pair Group Method with Arithmatic Mean (UPGMA), Neighbor Joining and Maximum Parsimony trees. For Neighbor Joining trees and UPGMA trees, the distances were calculated using the algorithm Kimura 2-parameter. For parsimony analysis, parsimonious trees were searched using close-neighbor-interchange (CNI) method. Bootstrap test was applied for 500 replications.
The spacers between the 5S rRNA genes are variable. This variation is high enough to separate Stemona from its adulterant and also to differentiate among different Stemona species.

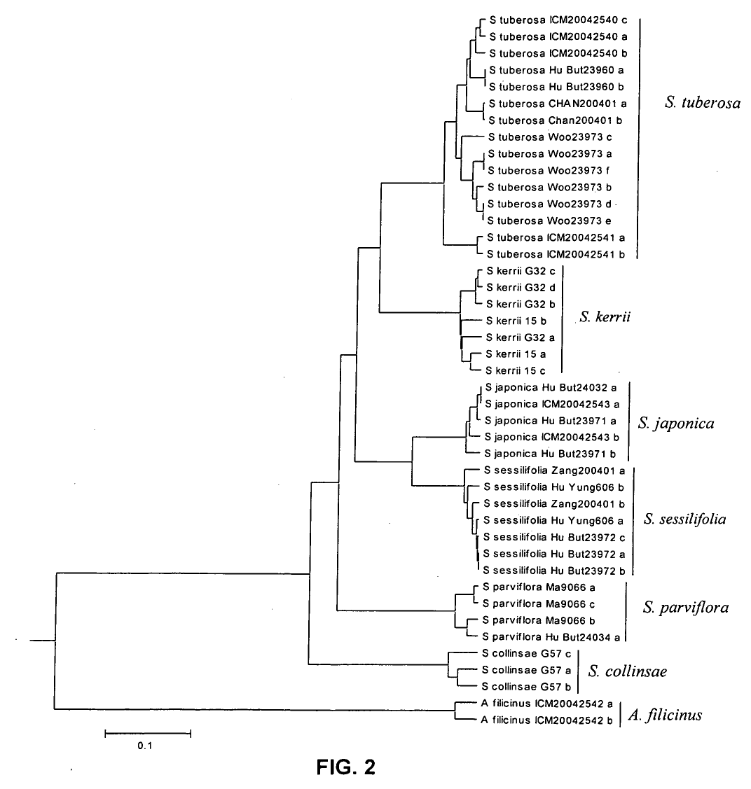

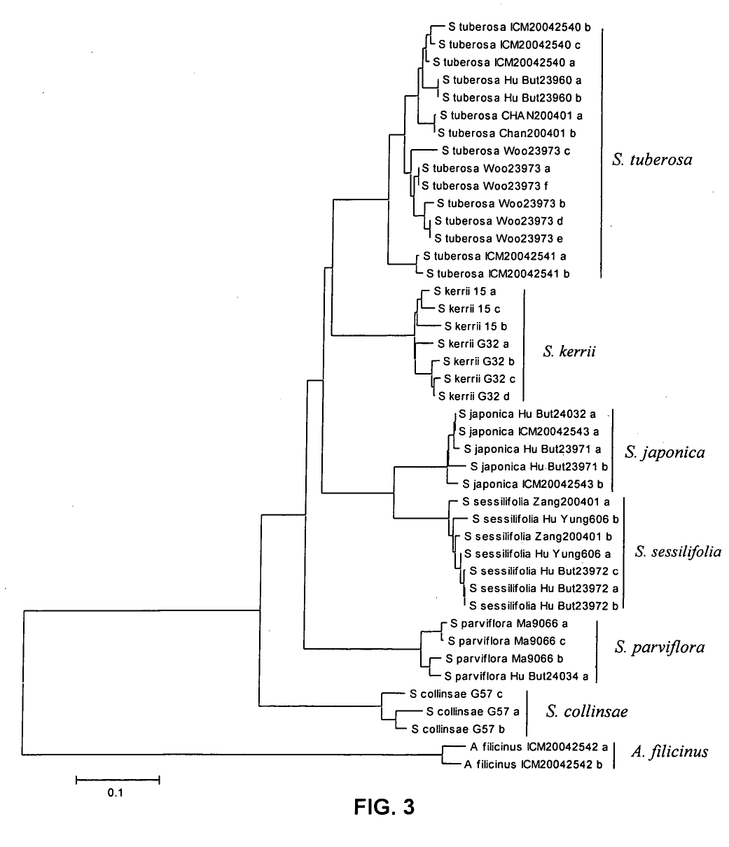

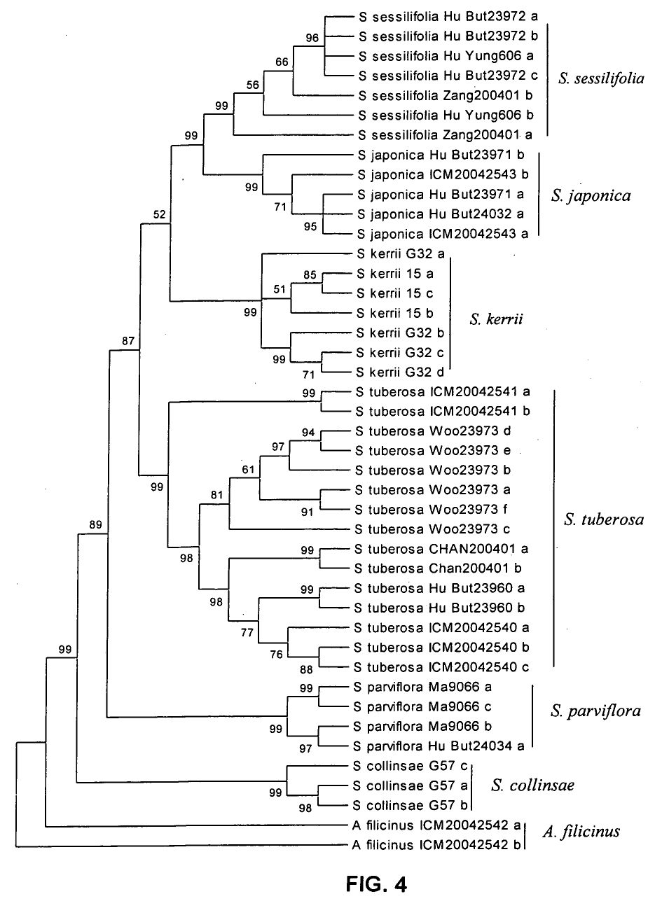

The 5S rRNA spacer sequences in Asparagus filicinus were highly different from Stemona species. Asparagus filicinus can be easily distinguished from Stemona species according to the difference in size of the 5S rRNA spacers, the low percentage similarity and its position in the phylogenetic trees. The size of the PCR product from Asparagus filicinus is 600 bp, while that of Stemona is 500 bp only. The 5S rRNA spacer sequences of Stemona and Asparagus are too different to prepare alignment. The percentage similarity between Asparagus filicinus and Stemona species is about 16% on average. In the UPGMA tree (shown in FIG. 2), Neighbour-Joining tree (shown in FIG. 3) and Maximum Parsimony tree (shown in FIG. 4) constructed, Asparagus filicinus does not group with the Stemona species.

The 5S rRNA spacer sequences of Stemona can also differentiate different Stemona species from one another. The result of sequence alignment 5 shows that the 300 bp-400 bp region of the spacer was the most variable region (see FIG.1). Within this variable region, each species has unique insertion and deletion sections or unique sequences. The spacer sequences are very conserve within the same species while the interspecific variation is very large. As is presented in Table 2, the intraspecific percentage similarity is about 90-100%. The interspecific percentage similarity among different Stemona species is about 70-80%. Thus, 5S rRNA spacers is a very useful molecular marker for authenticating Radix Stemonae. In all the phylogenetic trees constructed based on 5S rRNA spacers, six Stemona species formed a lade distinct from Asparagus filicinus. The lade of Stemona further branches out to four smaller clades representing four Stemona species. These clades have bootstraps value of 100 in the bootstraps test, which means these clades are well supported.

TABLE 2
Percentage Similarity of 5S rRNA Spacer Sequences
among six Stemona Species

| | Stemona | Stemona | Stemona | Stemona | Stemona | Stemona | Asparagus |
|---|---|---|---|---|---|---|---|
| | tuberosa | japonica | sessilifolia | parviflora | collinsae | kerrii | filicinus |
| Stemona | 89-100% | 73-79% | 76-81% | 75-78% | 71-75% | 78-81% | 16-19 |
| tuberosa | (94.5%) | (76%) | (78.5%) | (76.5%) | (73%) | (79.5%) | (17.5%) |
| Stemona | | 96-100% | 83-86% | 72-75% | 65-67% | 75-77% | 16-18% |
| japonica | | (98%) | (84.5%) | (73.5%) | (66%) | (76%) | (17%) |
| Stemona | | | 96-100% | 71-74% | 70-72% | 76-78% | 15-17% |
| sessilifolia | | | (98%) | (72.5%) | (71%) | (77%) | (16%) |
| Stemona | | | | 94-98% | 70-72% | 74-77% | 16% |
| parviflora | | | | (96%) | (71%) | (75.5%) | |
| Stemona | | | | | 93-95% | 69-74% | 15-16% |
| collinsae | | | | | (94%) | (71.5%) | (15.5%) |
| Stemona | | | | | | 93-99% | 13-16% |
| kerrii | | | | | | (96%) | (14.5%) |
| Asparagus | | | | | | | 95% |
| filicinus | | | | | | | |

As accomplished in the foregoing embodiment, the present invention utilizing DNA techniques and the genetic characteristics of the 5S rRNA spacer, provides a useful method in authenticating this genus of medicinal materials (herbs) and/or plants. As an example, Radix Stemonae can be authenticated readily and objectively from adulterants. By analyzing 5S rRNA spacer, it is also possible to identify herbal materials down to species level. It is contemplated that satisfactory results may be obtained by applying the foregoing authentication method based on the 5S rRNA spacer to other applications.

While there have been described and pointed out fundamental novel features of the invention as applied to a preferred embodiment thereof, it will be understood that various omissions and substitutions and changes, in the form and details of the processes and methods illustrated, may be made by those skilled in the art without departing from the spirit of the invention. For example, it is expressly intended that all combinations of those elements of method steps which perform substantially the same function in substantially the same way to achieve the same results are within the scope of the invention.

The invention is not limited by the embodiments described above which are presented as examples only but can be modified in various ways within the scope of protection defined by the appended patent claims.

We claim:

1. A method for authenticating an herb or a plant within the genus of Radix Stemonae, comprising the steps of:
(a) obtaining a sample of genomic DNA from said herb or plant comprising 5S rRNA;
(b) analyzing the DNA sequence of said 5S rRNA; and
(c) comparing said DNA sequence with a standard DNA sequence, with a portion of said standard DNA sequence, or with a DNA sequence that encompasses said standard DNA sequence or a portion of said standard DNA sequence; said standard DNA sequence being selected from the group consisting of: SEQ ID NO: 5, SEQ ID NO: 9, SEQ ID NO: 10, SEQ ID NO: 11, SEQ ID NO:12, SEQ ID NO: 13, SEQ ID NO: 14, SEQ ID NO: 15, SEQ ID NO:16, SEQ ID NO:17, SEQ ID NO:18, SEQ ID NO:19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, and SEQ ID NO: 47.

2. The method of claim 1, wherein prior to analyzing the DNA sequence of said 5S rRNA in step (b), said 5S rRNA spacer region of said genomic DNA is amplified.

3. The method of claim 1, wherein a portion of said standard DNA sequence is used and said portion accounts for 10%-100% of the length of said standard DNA sequence, said length being measured in terms of the number of DNA base pairs.

4. The method of claim 1, wherein said herb or plant is purportedly to be of Stemona japonica, Stemona sessilifolia or Stemona tuberosa.

5. The method of claim 2, wherein said 5S rRNA spacer region of said genomic DNA is amplified is performed with a PCR process.

6. The method of claim 5, wherein said PCR process uses two pairs of DNA primers which are SEQ ID NO: 1 and SEQ ID NO: 2 or SEQ ID NO: 3 and SEQ ID NO: 4.

7. The method of claim 1, wherein step (c) is performed by calculating similarity percentage between said DNA sequence with the DNA sequence of said standard.

8. The method of claim 1, wherein step (c) is performed by building a phylogenetic tree.

9. The method of claim 8, wherein said phylogenetic tree is generated by Neighbor Joining analysis or by Unweighted Pair Group Method with Arithmetic Mean.

10. The method of claim 1, wherein step (c) is performed through parsimony analysis by searching parsimonious trees using a close-neighbor-interchange method.
